(19) United States
(12) Patent Application Publication
(10) Pub. No.: US 2011/0097318 A1
(43) Pub. Date: Apr. 28, 2011

(54) Solid-State Protein Formulation

(75) Inventor: Himanshu Gadgil, Tarzana, CA (US)

(73) Assignee: AMGEN INC., Thousand Oaks, CA (US)

(21) Appl. No.: 12/672,852

(22) PCT Filed: Aug. 29, 2008
(86) PCT No.: PCT/US08/74793

§ 371 Date: Dec. 3, 2010

Related U.S. Application Data
(60) Provisional application No. 60/969,544, filed on Aug. 31, 2007.

(51) Int. Cl. A61K 39/395; A61K 38/00; A61P 43/00; A61M 5/31; B65B 3/04

(52) U.S. Cl. 424/130.1; 514/1.1; 604/82; 604/190; 141/2

(57) ABSTRACT

Provided are systems comprising delivery vehicles for the stable storage of immobilized proteins, e.g., protein therapeutics, in a form amenable to administration, such as by injection or infusion, in combination with an elution fluid. Also provided are proteins adsorbed to chromatography media in a form compatible with a one-step administration of the protein. Exemplary delivery vehicles are pre-filled syringes and pre-filled infusion modules; exemplary proteins are antibodies useful in therapy. Also provided are methods of producing the immobilized proteins and methods of using the immobilized proteins, e.g., protein therapeutics.

This application claims the priority benefit of U.S. Ser. No. 60/969,544, filed Aug. 31, 2007.

### FIELD

The disclosure relates generally to the field of therapeutic protein storage and delivery into patients.

### BACKGROUND

The primary structure of the individual peptide chains of all proteins, including proteins of therapeutic significance, is a series of amino acids, some of which have ionizable side groups, such as glutamate, aspartate, histidine, arginine, and lysine. The presence of these ionizable residues in a given protein influences the pI of that protein, or the pH at which the protein lacks a net overall charge. A wide variety of protein buffers have been known for some time, and these compositions protect proteins from pH changes of such magnitude that the stability of the proteins may be compromised. Nonetheless, buffers need not, and frequently do not, maintain the pH of a protein-containing composition precisely at the pI of that protein. Therefore, proteins are frequently maintained in moderately stable compositions buffered to pH values that leave the protein with a net charge. Although buffered protein solutions provide some stability to the protein, that protein is frequently measured in minutes at room temperature, and not in days, weeks or years. In addition, proteins in liquid form can be susceptible to shear-induced modifications. Another drawback of liquid formulations is the lower stability of proteins at high concentrations. Thus, buffered protein compositions do not provide a long-term answer to the question of how to stabilize commercially, e.g., therapeutically, active proteins.

Additionally, certain proteins cannot be stabilized in solution form for storage at ambient temperatures, for any significant period of time. Hence, many such proteins must be stored at low temperatures, frozen, or lyophilized. These solutions are inadequate as they add to the cost of storage and/or preparation and reduce convenience of use.

Thus, a need continues to exist in the art for the stable storage of proteins and peptides, including therapeutic proteins and peptides. Further, a need exists for a stable storage form that is convenient, inexpensive and readily adaptable to clinical use.

### SUMMARY

The subject matter described in detail herein provides a wholly new approach to stabilization, storage, and delivery of protein pharmaceuticals. That subject matter provides for stable storage of therapeutic proteins and peptides, such as therapeutic antibodies, by maintaining the proteins non-covalently bound to a chromatography medium, e.g., an ion exchange medium or media, while being readily elutable or dissociable from the medium or media for direct delivery of the proteins into patients, eliminating a need for storage of the proteins in a liquid form at ambient temperatures.

In one aspect, the disclosure provides a system for storing a protein, such as a protein therapeutic, in a stable form amenable, for example, to one-step administration thereof, the system comprising (a) a delivery vehicle comprising (i) at least one chamber in which is disposed a chromatography medium selected from the group consisting of a cation exchange medium, an anion exchange medium, an affinity medium and a hydrophobic interaction medium, wherein the medium is non-covalently bound to the protein, such as being bound to at least one therapeutically effective dose of a protein therapeutic; (ii) an inlet port; and (iii) a medium restrictor for substantially preventing discharge of the medium from the delivery vehicle; and (b) an elution fluid calibrated to release at least a portion, such as a therapeutically effective dose, of the protein (e.g., protein therapeutic). In some embodiments, the medium restrictor is selected from the group consisting of a filter and an outlet port. Exemplary outlet ports include an outlet port that comprises a valve for preventing discharge of the medium or an outlet port that comprises an outlet aperture sized to prevent discharge of the medium.

Any of a wide range of proteins, such as protein therapeutics, e.g., naturally occurring proteins, synthetic, non-naturally occurring, and/or fusion proteins such as peptibodies and avimers, and therapeutic protein fragments are suitable for inclusion in the delivery vehicle, including any form of an antibody (e.g., monoclonal or polyclonal, intact antibody or fragment thereof (Fab or F(ab′)₂) obtained from any animal or antibody-producing cell source, such as a mammal or mammalian cell, chimeric, humanized, and human antibodies of any isotype or mixed isotype, single-chain molecules including recombinant antibody forms and camelid antibodies, and the like. Beyond the various forms of antibody and antibody-like proteins, any kind of protein (including polypeptides and/or peptides) known in the art, whether naturally occurring or non-naturally occurring and whether synthetic or derived from a natural source, may be used in the delivery vehicle according to the disclosure, including but not limited to structural proteins, enzymes, hormones, growth factors, regulatory proteins including expression factors, chimeric and non-chimeric multi-chain proteins, single-chain proteins, fusion proteins such as Fc-fusion proteins such as peptibodies or avimers, and fragments, derivative or variants of any of these proteins.

In some embodiments, the protein therapeutic is selected from the group consisting of etanercept (Enbrel®, a TNF blocker), erythropoietin, darbepoetin alfa (Aranesp®, an EPO analog), filgrastim (Neupogen® or recombinant methionyl human granulocyte colony-stimulating factor (r-metHuG-CSF)) and pegfilgrastim (Neulasta®, a PEGylated filgrastim). Embodiments of the protein therapeutic also include therapeutic antibodies such as Humira (adalimumab), Synagis (palivizumab), 146B7-CHO (anti-IL15 antibody, see U.S. Pat. No. 7,153,507), vectibix (panitumumab), Rituxan (rituximab), zevalin (ibritumomab tiuxetan), anti-CD80 monoclonal antibody (mAb) (galiximab), anti-CD23 mAb (lumiliximab), M200 (volociximab), anti-Cripto mAb, anti-BR3 mAb, anti-IGF1R mAb, Tysabri (natalizumab), Daclizumab, humanized anti-CD20 mAb (ocrelizumab), soluble BAFF antagonist (BR3-Fc), anti-CD40L mAb, anti-TWEAK mAb, anti-IL5 Receptor mAb, anti-ganglioside GM2 mAb, anti-FGF8 mAb, anti-VEGFR/Flt-1 mAb, anti-ganglioside GD2 mAb, Actilyse® (alteplase), Metalyse® (tenecteplase), CAT-3888 and CAT-8015 (anti-CD22 dsFv-PE38 conjugates), CAT-354 (anti-IL13 mAb), CAT-5001 (anti-mesothelin dsFv-PE38 conjugate), GC-1008 (anti-TGF-β mAb), CAM-3001 (anti-GM-CSF Receptor mAb), ABT-874 (anti-IL12 mAb), Lymphostat B (Belimumab; anti-BlyS mAb), HGS-ETR1 (mapatumumab; human anti-TRAIL Receptor-1 mAb), HGS-ETR2 (human anti-TRAIL Receptor-2 mAb), ABthrax™ (human, anti-protective antigen (from B. anthracis) mAb), MYO-029 (human anti-GDF-8 mAb), CAT-213 (anti-eotaxin1 mAb), Erbitux, Epratuzumab, Remicade (infliximab; anti-TNF mAb), Herceptin® (traztusumab), Mylotarg (gemtuzumab ozogamicin), VECTIBLIX (panatumamab), ReoPro (abciximab), Actemra (anti-IL6 Receptor mAb), Avastin, HuMax-CD4 (zanolimumab), HuMax-CD20 (ofatumumab), HuMax-EGFr (zalutumumab), HuMax-Inflam, R1507 (anti-IGF-1R mAb), HuMax HepC, HuMax CD38, HuMax-TAC (anti-IL2Ra or anti-CD25 mAb), HuMax-ZP3 (anti-ZP3 mAb), Bexxar (tositumomab), Orthoclone OKT3 (muromonab-CD3), MDX-010 (ipilimumab), anti-CTLA4, CNTO 148 (golimumab; anti-TNFα Inflammation mAb), CNTO 1275 (anti-IL12/IL23 mAb), HuMax-CD4 (zanolimumab), HuMax-CD20 (ofatumumab), HuMax-EGFR (zalutumumab), MDX-066 (CDA-1) and MDX-1388 (anti-C. difficile Toxin A and Toxin B C mAbs), MDX-060 (anti-CD30 mAb), MDX-018, CNTO 95 (anti-integrin receptors mAb), MDX-1307 (anti-Mannose Receptor/hCGβ mAb), MDX-1100 (anti-IP10 Ulcerative Colitis mAb), MDX-1303 (Valortim™), anti-B. anthracis Anthrax, MEDI-545 (MDX-1103, anti-IFNα), MDX-1106 (ONO-4538; anti-PD1), NVS Antibody #1, NVS Antibody #2, FG-3019 (anti-CTGF Idiopathic Pulmonary Fibrosis Phase I Fibrogen), LLY Antibody, BMS-66513, NI-0401 (anti-CD3 mAb), IMC-18F1 (VEGFR-1), IMC-3G3 (anti-PDGFRα), MDX-1401 (anti-CD30), MDX-1333 (anti-IFNAR), Synagis (palivizumab; anti-RSV mAb), Campath (alemtuzumab), Velcade (bortezomib), MLN0002 (anti-alpha4beta7 mAb), MLN1202 (anti-CCR2 chemokine receptor mAb)., Simulect (basiliximab), prexige (lumiracoxib), Xolair (omalizumab), ETI211 (anti-MRSA mAb), IL-1 Trap (the Fc portion of human IgG1 and the extracellular domains of both IL-1 receptor components (the Type I receptor and receptor accessory protein)), VEGF Trap (Ig domains of VEGFR1 fused to IgG1 Fc), Zenapax (Daclizumab), Avastin (Bevacizumab), MabThera (Rituximab), MabTheraRA (Rituximab), Tarceva (Erlotinib), Zevalin (ibritumomab tiuxetan), Zetia (ezetimibe), Zyttorin (ezetimibe and simvastatin), Atacicept (TACI-Ig), NI-0401 (anti-CD3 in Crohn's disease), Adecatumumab, Golimumab (anti-TNFα mAb), Epratuzumab, Gemtuzumab, Raptiva (efalizumab), Cimzia (certolizumab pegol, CDP 870), (Soliris) Eculizumab, Pexelizumab (Anti-C5 Complement), MEDI-524 (Numax), Lucentis (Ranibizumab), 17-1A (Panorex), Trabio (lerdelimumab), TheraCim hR3 (Nimotuzumab), Omnitarg (Pertuzumab), Osidem (IDM-1), OvaRex (B43.13), Nuvion (visilizumab), and Cantuzamab. Other embodiments of the disclosure comprise a protein therapeutic that is not an antibody, such as a peptide hormone, a peptide ligand, signaling molecules such as cytokines and chemokines, or any protein known to exert a therapeutically beneficial effect, such as natrecor (nesiritide; rh type B natriuretic peptide) erythropoietin (see above), insulin, and the like.

In certain embodiments, the protein therapeutic has a pI of at least 7.0. More generally, considerations of the calculated or determined pI value of a protein and the pH range in which that protein is stable will guide selection of suitable loading and elution buffers as well as a suitable chromatography medium that is an ion exchange medium. For example, a protein with a pI of 7 that is stable at pH 7-9 could be loaded onto an anion exchange medium in a loading buffer at pH 8.0, at which pH the protein will have a net negative charge and behave as an anion. One of skill would recognize that the same protein could be loaded onto a cation exchange medium at a pH less than 7 (using a suitable loading buffer to maintain the desired pH) if the protein were stable enough at that pH to retain sufficient activity, e.g., therapeutic activity.

The system also includes a medium, which may be a hydrophobic interaction medium, an affinity chromatography medium, an anion exchange medium (ether weak or strong exchanger), such as a sulfopropyl-containing sorbent or base medium, or a cation exchange (weak or strong) medium, such as a carboxymethyl-, sulfopropyl-, or methyl sulfonate-containing sorbent or base medium.

To inhibit or prevent co-administration of the medium with the eluted protein therapeutic, in some embodiments the medium restrictor is a filter, such as an in-line filter, for preventing discharge of the medium, e.g., when administering at least one dose of a protein therapeutic. Also contemplated is an outlet port comprising a medium restrictor in the form of an outlet port aperture sized to prevent discharge of the medium.

According to certain embodiments of the system, the delivery vehicle may comprise a syringe, such as a syringe with one or more chambers, e.g., a single-chambered or a dual-chambered syringe. In dual-chambered syringes, the medium, whether bound to at least one dose of at least one protein therapeutic or not, is localized to one chamber. In syringes having more than two chambers, the medium remains localized to a single chamber, typically the chamber closest to the outlet port. In some embodiments of the system comprising a dual-chambered syringe, a pressure-sensitive barrier is placed between the two chambers to prevent fluid flow. The barrier is ruptured by an increase in pressure, such as would occur when the pressure of an elution fluid was raised by depressing the plunger of the syringe.

Contemplated within the system is an elution fluid that is physiologically compatible with a subject to which the protein, e.g., protein therapeutic, is to be administered.

A related aspect of the disclosure is a method of producing the system described above, comprising (a) adding at least a predetermined quantity of the medium to the chamber comprising the medium, wherein the medium is non-covalently bound to a protein, such as a protein therapeutic; and (b) determining the volume of an elution fluid to elute at least a portion of the protein, such as at least one therapeutically effective dose of the protein therapeutic. In some embodiments, the medium is a cation exchange medium and the protein (e.g., protein therapeutic) has a pI of at least 7.0. In some embodiments as well, e.g., where the delivery vehicle is a syringe or infusion module, contemplated is a method of producing the system described above, comprising adding an ion exchange medium in a buffer to a second chamber of the syringe or infusion module, wherein the ion exchange medium has a protein non-covalently bound, such as by having at least one dose of an ionizable protein therapeutic non-covalently bound, wherein the buffer has a pH different than the pI of the medium, and wherein the ion exchange medium in contact with the buffer is ionized.

Other methods of producing the system according to the disclosure comprise adding an ion exchange medium in a buffer to the second chamber of the delivery vehicle, e.g., syringe, wherein the ion exchange medium has a protein non-covalently bound, such as by having at least one therapeutically effective dose of an ionizable protein therapeutic non-covalently bound, wherein the buffer has a pH different than the pI of the medium and wherein the ion exchange medium in contact with the buffer is ionized, applying the first barrier between the first chamber and the second chamber, and adding an eluting buffer to the first chamber.

Another aspect of the disclosure is a delivery vehicle comprising (a) at least one chamber in which is disposed a chromatography medium selected from the group consisting of a cation exchange medium, an anion exchange medium, an affinity medium and a hydrophobic interaction medium, wherein the medium is non-covalently bound to at least one protein, such as by being non-covalently bound to at least one therapeutically effective dose of a protein therapeutic; (b) an inlet port; (c) an outlet port; and (d) a medium restrictor for substantially preventing discharge of the medium from the delivery vehicle. In certain embodiments, the protein is a protein therapeutic, and in some embodiments, the protein therapeutic is an antibody. Other proteins according to the disclosure include, but are not limited to, etanercept, erythropoietin, darbepoetin alfa, filgrastim and pegfilgrastim. The medium of the delivery vehicle may be a cation exchange medium, such as a cation exchange medium having a functional group selected from the group consisting of a carboxymethyl group, a sulfopropyl group and a methyl sulfonate. Some embodiments of the delivery vehicle comprise a filter, such as an in-line filter, for preventing discharge of the medium from the delivery vehicle, e.g., by preventing discharge of the medium from the chamber comprising the medium. Implementations of the delivery vehicle according to the disclosure have an outlet port that is sized to prevent discharge of the medium from the chamber comprising the medium.

In certain embodiments, the delivery vehicle is a syringe or an infusion module. The delivery vehicle (e.g., syringe or infusion module) may comprise two chambers, wherein the medium is localized to one chamber. In such embodiments, the delivery vehicle (syringe or infusion module) may further comprise a pressure-sensitive barrier separating the two chambers. Embodiments of the delivery vehicle are contemplated that comprise a medium that is non-covalently bound to at least one protein, such as by being bound to at least one therapeutically effective dose of a protein therapeutic. The delivery vehicle may further comprise a physiologically compatible elution fluid.

Another aspect of the disclosure is drawn to a method of administering a protein, such as a protein therapeutic, to a subject using the system or delivery vehicle described above, comprising (a) contacting the medium non-covalently bound to at least one protein, e.g., a therapeutically effective dose of a protein therapeutic, with an elution fluid; (b) eluting at least a portion of the protein, such as by eluting at least one therapeutically effective dose of the protein therapeutic; and (c) discharging the eluted protein, e.g., by discharging at least one therapeutically effective dose of the eluted protein therapeutic, from the delivery vehicle, thereby administering the protein, e.g., protein therapeutic, to the subject. The subject may be any animal in need of a protein such as a protein therapeutic, including any mammal, such as man, domesticated livestock, pets, and the like. In a related aspect, the disclosure provides a method of administering a protein (e.g., protein therapeutic) to a subject, comprising (a) contacting a medium non-covalently bound to at least one protein, such as by contacting at least one therapeutically effective dose of a protein (e.g., protein therapeutic) with an elution fluid, wherein the medium is confined in one chamber of a syringe or infusion module comprising at least one chamber; (b) eluting at least a portion of the protein, such as by eluting at least one therapeutically effective dose of the protein therapeutic; and (c) discharging the eluted protein (e.g., protein therapeutic) from the syringe or infusion module, thereby administering the portion of the protein, such as a therapeutically effective dose of the protein therapeutic, to the subject.

In certain embodiments, the protein, e.g., therapeutic protein, is an antibody. In some embodiments, the contacting step comprises rupturing a fluid-impermeable barrier covering the inlet port of the chamber comprising the medium. Rupturing the barrier may be accomplished by any method known in the art. It is expressly contemplated in some embodiments of the method of administering a protein that the system will further comprise a syringe plunger comprising a head member sealingly engaged with the internal surface of the syringe. In such embodiments, rupturing is accomplished by applying fluid pressure to the membrane by actuating the syringe plunger. In some embodiments, the fluid-impermeable barrier will be ruptured by a projection capable of piercing or weakening the barrier, e.g., by projecting from a syringe plunger head through sufficient fluid in chamber 2 to contact the barrier prior to rupture due to fluid pressure increase alone. Barrier rupture may be achieved by the combined effect of a syringe plunger head projection contacting and partially disrupting the barrier along with the effect attributable to increased fluid pressure on the barrier attending syringe plunger actuation. In each of the methods of administering the protein therapeutic described in this paragraph, the protein therapeutic may be an antibody or it may be selected from the group consisting of etanercept, erythropoietin, darbepoetin alfa, filgrastim and pegfilgrastim.

Another aspect according to the disclosure is a kit for administering a protein comprising an infusion module or syringe, wherein the infusion module or syringe comprises a chromatography medium non-covalently bound to a protein, and a package insert for providing instruction on the use thereof.

Yet another aspect according to the disclosure is a use of a chromatography medium non-covalently bound to a protein in the preparation of a medicament for the treatment of a disease.

Other features and advantages of the invention will be better understood by reference to the brief description of the drawing and the detailed description of the invention that follow.

### BRIEF DESCRIPTION OF THE DRAWING

While the specification concludes with claims particularly pointing out and distinctly claiming the subject matter that is regarded as the present invention, it is believed that the invention will be more fully understood from the following description taken in conjunction with the accompanying drawings. Some of the figures may have been simplified by the omission of selected elements for the purpose of more clearly showing other elements. Such omissions of elements in some figures are not necessarily indicative of the presence or absence of particular elements in any of the exemplary embodiments, except as may be explicitly delineated in the corresponding written description. None of the drawings are necessarily to scale. Throughout, a numbering convention has been adopted such that similar features of the various embodiments have been numbered in a similar manner.

FIG. 1 shows an embodiment of a delivery vehicle according to the disclosure, the delivery vehicle comprising a syringe comprising at least one chamber in which is disposed a chromatography medium non-covalently bound to a protein, an inlet port, an outlet port and a medium restrictor.

FIG. 2 illustrates another embodiment of a delivery vehicle comprising a syringe according to the disclosure.

FIG. 3 depicts another embodiment of a delivery vehicle comprising a syringe according to the disclosure.

FIG. 4 reveals yet another embodiment of a delivery vehicle comprising a syringe according to the disclosure.

FIG. 5 provides another embodiment of a delivery vehicle comprising a syringe according to the disclosure.

FIG. 6 shows an embodiment of a syringe plunger according to the disclosure.

FIGS. 7a-d illustrates various embodiments of a syringe plunger head according to the disclosure.

FIG. 8 shows an embodiment of a delivery vehicle comprising an infusion module according to the disclosure, the infusion module comprising at least one chamber in which is disposed a chromatography medium non-covalently bound to a protein.

FIG. 9 reveals another embodiment of a delivery vehicle comprising an infusion module according to the disclosure.

FIG. 10a depicts another embodiment of a delivery vehicle comprising an infusion module according to the disclosure, while FIG. 10b shows a pestle member suitable for use in rupturing or breaking the barrier contained within the delivery vehicle.

FIG. 11 provides yet another embodiment of a delivery vehicle comprising an infusion module according to the disclosure.

FIG. 12 illustrates still another embodiment of a delivery vehicle comprising an infusion module according to the disclosure.

FIG. 13 shows an embodiment of a packet according to the disclosure, the packet comprising a sealed perimeter defining a packet interior containing a chromatography medium non-covalently bound to a protein and optionally containing a region of the sealed perimeter that is more frangible than the rest of the perimeter.

FIG. 14 depicts another embodiment of a packet according to the disclosure.

FIG. 15 provides a schematic illustration of an embodiment of a delivery vehicle comprising a dual-chambered syringe suitable for long-term therapeutic protein storage and one-step administration of the therapeutic. A first chamber comprises a cation exchange medium denoted by the circles, which are negatively charged. Y-shaped structures refer to the protein, which has a net positive charge. An outlet port comprising a filter is provided to retain the chromatography medium. FIG. 15a provides cation exchange medium non-covalently bound to protein introduced into the first chamber comprising the medium using an acidic buffer imparting positive charge to the protein. FIG. 15b provides for the elution of protein using a buffer of higher pH (e.g., pH 7.0) showing eluted protein and retained cation exchange chromatography medium.

FIG. 16 provides a protein gel revealing that an exemplary protein, i.e., an agonistic anti-Tumor Necrosis Factor (TNF)-Related Apoptosis-Inducing Ligand (TRAIL) Receptor-2 antibody (an anti-TR2 antibody such as the antibodies described in provisional U.S. Ser. No. 60/713,433, filed Aug. 31, 2005, and provisional U.S. Ser. No. 60/713,478, filed Aug. 31, 2005 in 10 mM sodium acetate (pH 5), can be bound to carboxymethyl-sepharose, a weak cation exchange resin (WCX), and eluted using Tris-HCl, pH 8.0.

FIG. 17 provides two graphs showing reversed-phase chromatographic fractionations of the agonistic anti-TRAIL-R2 (anti-TR2) antibody described in connection with FIG. 16 bound to CM-sepharose and incubated in a shaker at 700 rpm at room temperature for three days as a form of short-term shear stress. Proteins were applied to the reversed-phase chromatography column at 2 mg/ml in 10 mM acetate, 5 mM sorbate, pH 5. The upper tracing of FIG. 17a: the agonistic anti-TRAIL-R2 antibody non-covalently bound to carboxymethyl-sepharose; the lower tracing of FIG. 17b: the agonistic anti-TRAIL-R2 antibody liquid formulation.

FIG. 18 shows a comparative gel electrophoretogram of the agonistic anti-TRAIL-R2 antibody described in connection with FIG. 16 in liquid formulation (A5Su) or non-covalently bound to CM-sepharose as described for FIG. 17. “Clips” refers to lower molecular weight degradation fragments of the agonistic anti-TRAIL-R2 antibody. The electrophoretogram shows greater degradation of the agonistic anti-TRAIL-R2 antibody in a liquid formulation relative to the CM-sepharose-bound formulation.

FIG. 19 provides graphs showing reversed-phase chromatographic fractionations of the agonistic anti-TRAIL-R2 antibody incubated as described above for FIG. 17 to induce short-term shear stress and then reduced using conventional techniques to hydrolyze the disulfide bonds characteristic of whole antibodies. FIG. 19a: graph for the agonistic anti-TRAIL-R2 antibody non-covalently bound to CM-sepharose during the short-term shear stress. FIG. 19b: graph for the agonistic anti-TRAIL-R2 antibody maintained in a liquid formulation for the short-term shear stress.

FIG. 20 shows a more detailed set of the graphs presented in FIG. 19 and described above. FIG. 20a shows the reversed-phase graph of the agonistic anti-TRAIL-R2 antibody described in connection with FIG. 16 subjected to short-term shear stress when non-covalently bound to CM-sepharose. FIG. 20b shows the reversed-phase graph of the agonistic anti-TRAIL-R2 antibody maintained in a liquid formulation during the short-term shear stress. More apparent in this detailed view are the lower molecular weight degradation products of the agonistic anti-TRAIL-R2 antibody found in the liquid formulation that are reduced or missing in the solid-state formulation of the agonistic anti-TRAIL-R2 antibody. A schematic illustration of the agonistic anti-TRAIL-R2 antibody is provided on the left side of the figure, correlating degradation products to peaks in the graphs as indicated.

FIG. 21 provides the results of ion exchange chromatography of an IgG1 designated herein as 146B7-CHO, demonstrating that modified and unmodified forms thereof can be discriminated. The 146B7-CHO antibody is a fully human anti-IL-15 monoclonal antibody expressed and purified from CHO cells and whose amino acid sequences are derived from 146B7, which is disclosed in U.S. Pat. No. 7,153,507, incorporated by reference herein in its entirety.

### DETAILED DESCRIPTION

The systems, delivery vehicles, and methods disclosed herein provide a coordinated approach to the stable, relatively long-term storage of proteins, such as therapeutic proteins, in a form amenable to delivery or administration to an animal in need. Proteins are non-covalently bound to a chromatography medium in a delivery vehicle, thereby stabilizing the protein for storage while providing the protein in a form readily prepared for administration by elution from the chromatography medium. As a consequence, proteins, such as therapeutic antibodies, receptors, peptide agonists/antagonists, and the like are available in a convenient, low-cost form with reduced waste due to activity loss upon storage. Accordingly, proteins for administration will be more affordable and will be amenable to more decentralized distribution, facilitating improved health care for man and animal in remote as well as urbanized environments.

An understanding of the substance of the disclosure will be facilitated by a consideration of the following express definitions of terms used herein. Unless a term is expressly defined herein by using a sentence that relates a term to its meaning, typically by expressly reciting the term, the word “means,” and then the definition, or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning.

### DEFINITIONS

“Administering” is given its ordinary and customary meaning of delivery by any suitable means recognized in the art. Exemplary forms of administering include oral delivery, anal delivery, direct puncture or injection, including intravenous, intraperitoneal, intramuscular, subcutaneous, intratumoral, and other forms of injection, gel or fluid application to an eye, ear, nose, mouth, anus or urethral opening not involving a solid-state carrier such as a microsphere or bead, and cannulation. A preferred mode of administration is injection by syringe, typically a needle-bearing syringe.

An “effective dose” is that amount of a substance that provides a beneficial effect on the organism receiving the dose and may vary depending upon the purpose of administering the dose, the size and condition of the organism receiving the dose, and other variables recognized in the art as relevant to a determination of an effective dose. The process of determining an effective dose involves routine optimization procedures that are within the skill in the art. The “loaded” syringes according to the disclosure comprise at least one dose of a protein therapeutic.

An “animal” is given its conventional meaning of a non-plant, non-protist living being. A preferred animal is a mammal, such as a human.

“Ameliorating” means reducing the degree or severity of, consistent with its ordinary and customary meaning.

“Pharmaceutical composition” means a formulation of compounds suitable for therapeutic administration, to a living animal, such as a human patient. Typical pharmaceutical compositions comprise a therapeutic agent such as an immunoglobulin-based therapeutic, in combination with an adjuvant, excipient, carrier, or diluent recognized in the art as compatible with delivery or administration to an animal, e.g., a human. Pharmaceutical compositions do not include therapeutics bound to solid carriers, such as microspheres, beads, ion exchange media and the like. The term “pharmacologically active” means that a substance so described is determined to have activity that affects a medical parameter (e.g., blood pressure, blood cell count, cholesterol level) or disease state (e.g., cancer, inflammatory disorders).

“Adjuvants,” “excipients,” “carriers,” and “diluents” are each given the meanings those terms have acquired in the art. An adjuvant is one or more substances that serve to prolong the immunogenicity of a co-administered immunogen. An excipient is an inert substance that serves as a vehicle, and/or diluent, for a therapeutic agent. A carrier is one or more substances that facilitates manipulation of a substance (e.g., a therapeutic), such as by translocation of a substance being carried. A diluent is one or more substances that reduce the concentration of, or dilute, a given substance exposed to the diluent.

“Media” and “medium” are used to refer to cell culture medium and to cell culture media throughout the application. As used herein, “media” and “medium” may be used interchangeably with respect to number, with the singular or plural number of the nouns becoming apparent upon consideration of the context of each usage.

“Substantially homogeneous” as used herein with reference to a preparation as disclosed herein means that the preparation includes a single species of a therapeutic compound detectable in the preparation of total therapeutic molecules in the preparation, unless otherwise stated at a specific percentage of total therapeutic molecules. In general, a substantially homogeneous preparation is homogeneous enough to display the advantages of a homogeneous preparation, e.g., ease in clinical application in predictability of lot to lot pharmacokinetics.

“Bioefficacy” refers to the capacity to produce a desired biological effect. Bioefficacy of different compounds, or different dosages of the same compound, or different administrations of the same compound are generally normalized to the amount of compound(s) to permit appropriate comparison.

The term “treatment” or “treating” includes the administration, to a subject in need, of an amount of a compound that will inhibit, decrease or reverse development of a pathological condition.

As used herein, the term “subject” is intended to mean a human or other mammal, exhibiting, or at risk of developing a deleterious disease, disorder or condition.

In general, “salt” refers to a salt form of a free base compound, as would be understood by persons of ordinary skill in the art. Salts may be prepared by conventional means, known to those skilled in the art. In general, “pharmaceutically-acceptable,” when used in reference to a salt, refers to salt forms of a given compound, which are within governmental regulatory safety guidelines for ingestion and/or administration to a subject. The term “pharmaceutically-acceptable salts” embraces salts commonly used to form alkali metal salts and to form addition salts of free acids or free bases. The nature of the salt is not critical, provided that it is pharmaceutically acceptable. The term “physiologically acceptable salts” comprises any salt or salts that are known or later discovered to be pharmaceutically acceptable. Some specific examples are: acetate; trifluoroacetate; hydrohalides, such as hydrochloride and hydrobromide; sulfate; citrate; tartrate; glycolate; and oxalate.

A “delivery vehicle” is a device for providing a substance, such as a protein therapeutic, to a subject such as an animal or human patient. Delivery vehicles generally contain the substance, such as a protein, and also provide the capacity to discharge the substance. Delivery vehicles include, but are not limited to, syringes comprising at least one chamber and infusion modules comprising at least one chamber.

### General Delivery System

### Delivery Vehicle

Delivery systems according to the disclosure provide a delivery vehicle and an elution fluid. The delivery vehicle provides a convenient device for the stable storage of a protein, such as a therapeutic protein, in a form amenable to convenient delivery of the protein to an animal subject. The delivery vehicle comprises at least one chamber, wherein the chamber contains a chromatography medium non-covalently bound to a protein, such as a protein therapeutic, an inlet port, an outlet port, and a medium restrictor. Any device known in the art as suitable for delivering a protein to a subject such as a human or other animal subject is contemplated, including a syringe or an infusion module, e.g., an infusion module suitable for incorporation into an intravenous delivery system. Delivery vehicles according to the disclosure include single-chambered, dual-chambered and multi-chambered syringes, with inter-chamber barriers designed to influence fluid communication between or among chambers of the delivery vehicle. Delivery vehicles may be glass, plastic, metal (e.g., stainless steel), or any composition known in the art as being compatible with the function of a delivery vehicle in delivering a compound to an animal subject. Although delivery vehicles may be generally cylindrical in overall shape, no significance is attached to such a shape and delivery vehicles of alternative overall shapes are contemplated.

Also comprehended by the subject matter disclosed herein are autoinjectors. The EpiPen® is an autoinjector that contains a spring-loaded needle that shoots through a membrane in the tip and into the recipient's body to deliver the medication, typically epinephrine to treat anaphylactic shock. A non-sterile, single-dose, hidden-needle autoinjector commercially available to administer β-interferon is the Rebiject™. Like the EpiPen®, the Rebiject™ is a spring-loaded device. Also available is the Adrenalina autoinjector, which provides an intuitive two-step safe activation procedure that can be performed with one hand. The Adrenalina autoinjector also uses an air-actuated plunger system to automate needle insertion and removal after a pre-set duration. This timing feature is useful in the devices of the disclosure in which elution fluid is brought into contact with the medium non-covalently bound to a protein prior to injection of the eluent. A multi-dose variant of the single-dose autoinjector is the Twinject™, which also contains a spring-loaded needle that shoots through a membrane in the tip and into the recipient's body to deliver the medication. A variation on the Twinject™ concept would provide a device with a plunger that punctured a membrane separating a first chamber containing chromatography medium non-covalently bound to a protein and a second chamber comprising an elution fluid. A period of time would then be allowed to pass (e.g., 5-15 seconds), and then the device could be forcefully applied to an area of the body of a subject, such as a thigh or buttocks, resulting in release of a spring-loaded mechanism for both inserting the needle and discharging fluid therethrough. Multi-dose capacity in an autoinjector is also useful in the delivery vehicles according to the disclosure. Suitable autoinjectors suitable for use as delivery vehicles, or for use in the systems and methods of the disclosure, as well as their construction and use, are described in U.S. Pat. Nos. 5,085,642, 5,102,393, 6,270,479, 6,371,939, and 7,118,553, each of which is incorporated herein in its entirety. Autoinjectors according to the disclosure may be driven by gas, electricity, an electro-mechanical mechanism or a mechanical mechanism, preferably a mechanical mechanism using an elastic material for storage and release of energy, e.g., a spring. The autoinjectors will provide for autoinsertion and autoinjection, and may provide for autoretraction (i.e., autoreturn).

A medium restrictor is a component of the delivery system that substantially prevents discharge of the chromatography medium, and may be a filter of suitable pore size or an outlet port of suitable pore size (i.e., aperture) or an outlet port comprising a valve useful in selectively permitting passage of an eluent containing a desorbed protein and inhibiting, or not permitting, passage of a chromatography medium. The inlet port, like the medium restrictor comprising an outlet port, of the delivery vehicle may be a fixed aperture or a controllable aperture, such as would be provided by a valve. In certain embodiments, a medium restrictor allowing passage of relatively large particles is used with a cross-linked chromatography medium unable to efficiently pass through the restrictor. The chromatography medium contained within a chamber of the delivery vehicle is an ion exchange medium, such as a cation or anion exchange medium, an affinity medium or a hydrophobic interaction medium. Any of a wide variety of proteins may be non-covalently bound, e.g., by ionic bonds, hydrogen bonds, van der Waals forces, and the like, to the chromatography medium. Exemplary proteins include therapeutic proteins, such as proteins or peptides derived from any form of an antibody, peptide hormones, peptide ligands, signaling molecules (e.g., cytokines, chemokines), and the like.

### Elution Fluid

In addition to the delivery vehicle, the delivery system according to the disclosure comprises an elution fluid. Elution fluids will be physiologically compatible with at least one animal subject, but it is understood that physiological compatibility may be achieved in part through dilution of the elution fluid upon administration. Elution fluids will also be capable of substantially dissociating a non-covalently bound protein from a chromatography medium. Suitable elution fluids will vary dependent upon the nature of the chromatography medium and, to some extent, dependent on the nature of the non-covalently bound protein. For example, in embodiments in which an ion exchange chromatography medium is used, an elution fluid may be a buffer of a particular pH and/or ionic strength.

### Delivery System Variants

In the following description of various embodiments according to the disclosure, it is understood that features shown for a given embodiment are generally appropriate for other embodiments of that aspect of the disclosure unless specifically and expressly excluded by the disclosure. In addition, similar features are identified by similar numbering in the figures of the drawing.

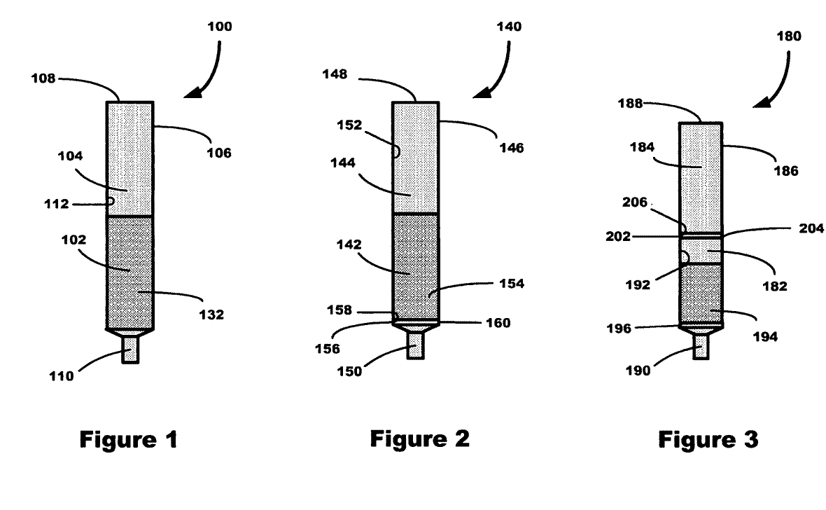

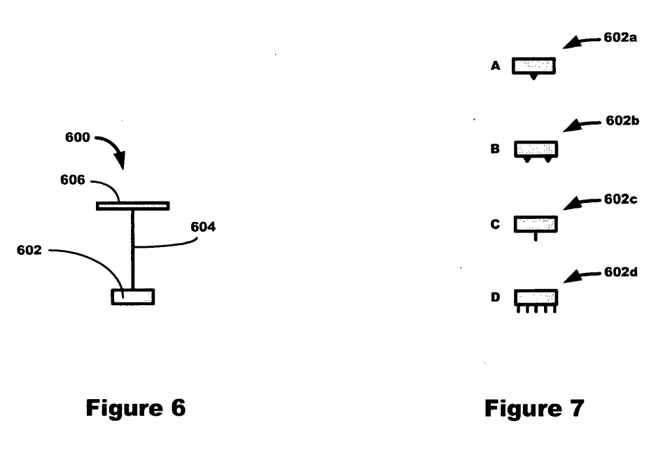

An embodiment of a delivery vehicle according to the disclosure is illustrated in FIG. 1, which shows a delivery vehicle in the form of a syringe 100 for containing a chromatography medium 132 non-covalently bound to a protein therapeutic. A surface or edge of chromatography medium 132 defines a boundary of first chamber 102 of syringe 100, wherein the surface or edge may be regular or irregular. Chromatography medium 132 may be an ion exchange medium, an affinity medium, or a hydrophobic interaction medium. A second chamber 104 of syringe 100 is defined by a surface or edge of chromatography medium 132, inner wall surface 112 of syringe 100, and inlet port 108. A syringe wall, and thus an outer wall surface 106 of syringe 100, is typically cylindrical and syringe 100 may be glass, plastic or any substance known in the art to be useful for forming syringes. At one end of syringe 100 is inlet port 108 through which material (e.g., fluid, chromatography medium 132) may enter syringe 100 and at the other end of syringe 100 is outlet port 110 through which material (e.g., fluid) may exit syringe 100. A plunger 600 suitable for use with syringe 100, or other syringes according to the disclosure, is illustrated in FIG. 6. Plunger 600 is composed of plunger head 602 connected to plunger shaft 604, which is, in turn, connected to plunger platen 606. Plunger head 602 slidably engages inner wall surface 112 of syringe 100.

Another embodiment of the syringe according to the disclosure is shown in FIG. 2, which provides syringe 140 having a first chamber 142 defined by chromatography medium 154 and a second chamber 144 defined by a surface or edge of chromatography medium 154, an inner wall surface 152, and inlet port 148. Syringe 140 also has inlet port 148, outlet port 150, and an outer wall surface 146. Interposed between first chamber 142 and outlet port 150 is outlet filter 156 for substantially retaining chromatography medium 154. In certain embodiments, outlet filter 156 retains all of chromatography medium 154 within syringe 140. In certain embodiments, outlet filter 156 prevents passage of a living cell, e.g., a bacterial cell, thereby providing a sterilizing function for fluid entering outlet port 150. Certain embodiments provide for an outlet filter 156 that prevents passage of virus particles, thereby providing for a virus-free fluid entering outlet port 150. Outlet filter 156 has an outer edge 160 that contacts a mating surface 158 of inner wall surface 152 of syringe 140. Outer edge 160 may form a press-fit with mating surface 158, or the edge and surface may be adhered to each other using any method known in the art, such as by use of a biocompatible adhesive applied to outer edge 160 and/or mating surface 158, or by heat-mediated fusion, depending on the composition of outer edge 160 and mating surface 158 of inner wall surface 152.

Still another embodiment of the syringe is shown in FIG. 3, which illustrates a syringe 180 having an inlet port 188, an outlet port 190, an outer wall surface 186, a first chamber 182 defined by an inner wall surface 192 of syringe 180, an outlet filter 196, and a barrier 202. First chamber 182 contains a chromatography medium 194, but chamber 182 is not defined by the volume of chromatography medium 194 contained within syringe 180 and, thus, chamber 182 may have a void volume or volume not occupied by chromatography medium 194, in addition to having a volume in which chromatography medium 194 is disposed. A second chamber 184 is defined by barrier 202, inner wall surface 192, and inlet port 188.

Barrier 202 separating first chamber 182 and second chamber 184 has the capacity to influence or affect fluid communication, e.g., fluid transmission, from or between first chamber 182 and second chamber 184. Barrier 202 may comprise a ruptureable or non-ruptureable frangible member, e.g., a thin layer or piece of plastic, rubber, ceramic, glass, or the like, or a pressure-sensitive member, e.g., a membrane in which fluid permeability varies positively with pressure. Barrier 202 has a circumferential face 204 that contacts a barrier-adhering region 206 of inner wall surface 192 of syringe 180 to effect a fluid barrier. Circumferential face 204 may form a press-fit with barrier-adhering region 206, or the face and region may be adhered to each other using any method known in the art, such as by use of a biocompatible adhesive applied to circumferential face 204 and/or barrier-adhering region 206, or by heat-mediated fusion, depending on the composition of circumferential face 204 and barrier-adhering region 206 of inner wall surface 192.

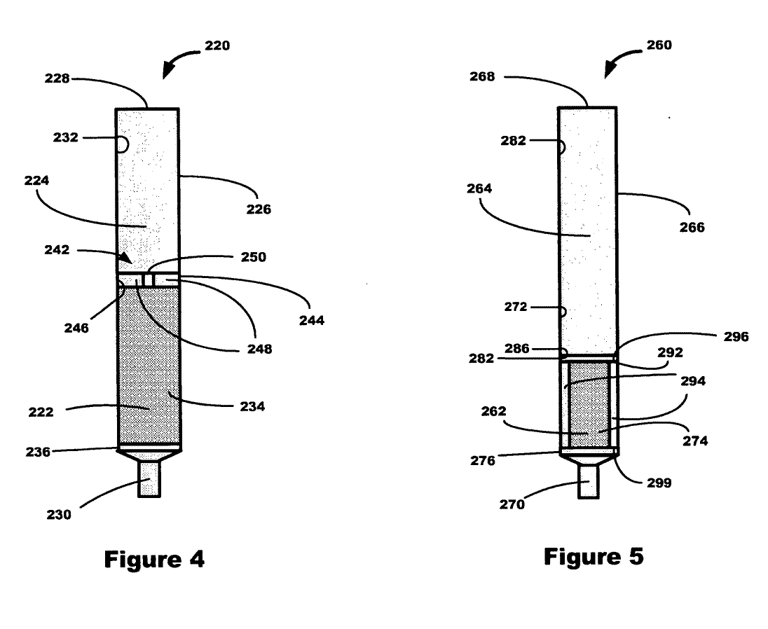

Another embodiment of the syringe according to the disclosure is shown in FIG. 4, wherein syringe 220 has a first chamber 222 and a second chamber 224, an outer wall surface 226, an inner wall surface 232, an inlet port 228, an outlet port 230, a barrier 242, and an outlet filter 236. First chamber 222 is defined by outlet filter 236, inner wall surface 232, and barrier 242, while second chamber 224 is defined by barrier 242, inner wall surface 232, and inlet port 228. As illustrated in FIG. 4, barrier 242 has a base member 248 and at least one pre-channel 250, defined as a region of barrier 242 structured to become a preferential channel for fluid flow, for example by being thinner and thus more prone to loss of barrier integrity than base material 248, by being made of a different material than base material 248, wherein the difference makes it easier to form a patent fluid channel through pre-channel 250 than through base material 248, by being geometrically structured to facilitate barrier breach upon an actuating event, such as by focusing the force accompanying depression of a syringe plunger (see, e.g., FIGS. 6a-d), and the like.

Yet another embodiment of the syringe according to the disclosure is provided in FIG. 5, wherein a syringe 260 has an outer wall surface 266, an inner wall surface 272, a first chamber 262, a second chamber 264, an inlet port 268, an outlet port 270, an outlet filter 276 and a barrier 282. In syringe 260, first chamber 262 has, at least in part, a smaller cross-sectional dimension than second chamber 264, because of the presence of a circumferential member 294. An edge or shoulder 296 of circumferential member 294 opposed to edge 299 in contact with syringe 260 (e.g., either outlet port 270 or outlet filter 272) is disposed in proximity to contact are 292 of barrier 282. Contact area 292 may passively rest on shoulder 296, e.g., when barrier 282 is press-fit into syringe 260. Contact area 292 may be adhered to shoulder 296 using any biocompatible adhesive known in the art, using heat-mediated fusion, or using any other method known in the art to be suitable for adhering the materials of contact area 292 and shoulder 296. The circumferential member 294 may be created by delivering a circumferential insert through syringe 260 until it is at the appropriate relative position along the generally cylindrical dimension of syringe 260, or until it seats on either outlet filter 276 or outlet port 270. The insert may be a press-fit or may be adhered to syringe 260 and/or outlet filter 276. In certain embodiments, circumferential member 294 is generated integrally with syringe 260. In certain embodiments, circumferential member 294 and syringe 260 are generally cylindrical and may be substantially co-axial in orientation.

As noted above, the embodiments of FIGS. 3-5 include a barrier that prevents transmission of material (e.g., fluid) between the first chamber and the second chamber. In certain embodiments, a plunger in the form illustrated in FIG. 6 is sufficient to cause transmission across the barrier by causing an increase in the differential pressure across the barrier sufficient to result in partial or complete loss of barrier function. According to other embodiments, however, this approach is insufficient or not desired and, in such embodiments, the plunger will have a plunger head capable of penetrating, scoring or otherwise weakening the barrier at one or more locations (see, e.g., FIGS. 7a-d). Either alone or in conjunction with the increased pressure differential resulting from actuation of the plunger, the plunger head projections will contribute to loss of barrier function.

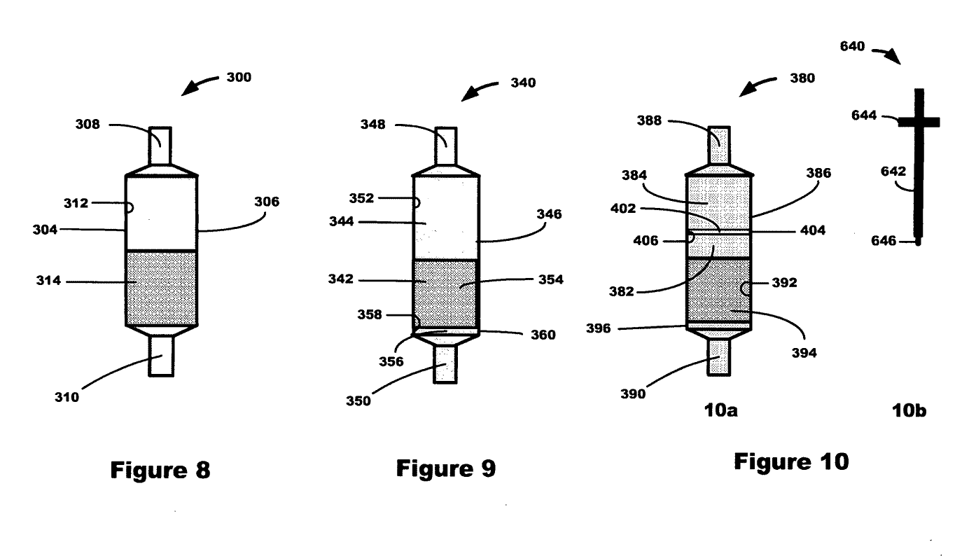

Another delivery vehicle according to the disclosure is an infusion module for confining a chromatography medium to which a protein, such as a protein therapeutic, is non-covalently bound. FIG. 8 illustrates an embodiment of infusion module 300 having an outer wall surface 306, a first chamber 302 defined by a regular or irregular surface of chromatography medium 314 non-covalently bound to the protein therapeutic, inner wall surface 312 and outlet port 310, a second chamber 304 defined by the regular or irregular surface of chromatography medium 314, inner wall surface 312, and inlet port 308. The volume of second chamber 304 is essentially the void volume of infusion module 300 (i.e., the total volume of infusion module 300 less the volume of chromatography medium 314). In certain embodiments, chromatography medium 314 is structured to limit passage through outlet port 310. Infusion modules according to the disclosure are suitable for use in administering a protein therapeutic by infusion, such as via an intravenous delivery system, as would be known in the art. When so arranged, an infusion module may be in direct or indirect fluid communication with a filter for limiting the flow of chromatography medium 314.

Another embodiment of the infusion module according to the disclosure is shown in FIG. 9, wherein an infusion module 340 has an outer wall surface 346, a first chamber 342 defined by a surface or edge of a chromatography medium 354, an inner wall surface 352, and an outlet filter 356, a second chamber 344 defined by the surface or edge of chromatography medium 354, inner wall surface 352 and inlet port 348, the aforementioned inlet port 348, outlet port 350, and outlet filter 356. In certain embodiments, outlet filter 356 has the property or properties of outlet filter 156 (see above) of the embodiment of the syringe illustrated in FIG. 2. In brief, outlet filter 356 may retain all of the chromatography medium within syringe 340. Additionally, outlet filter 356 may prevent passage of a living cell, e.g., a bacterial cell, thereby providing a sterilizing function for fluid entering outlet port 350. Certain embodiments provide for an outlet filter 356 that prevents passage of virus particles, thereby providing for a virus-free fluid entering outlet port 350. Inner wall surface 352 has a mating surface 358 that contacts an outer edge 360 of outlet filter 356. Outer edge 360 may form a press-fit with mating surface 358, or the edge and surface may be adhered to each other using any method known in the art, such as by use of a biocompatible adhesive applied to outer edge 360 and/or mating surface 358, or by heat-mediated fusion, depending on the composition of outer edge 360 and mating surface 358.

Yet another embodiment of the infusion module according to the disclosure is illustrated in FIG. 10, wherein infusion module 380 is shown to have an outer wall surface 386, a first chamber 382 containing a chromatography medium 394 non-covalently bound to a protein therapeutic, a second chamber 384, an inlet port 388, an outlet port 390, an outlet filter 396 and a barrier 402 interposed between first chamber 382 and second chamber 384. Barrier 402 may be a frangible member, e.g., a thin layer or piece of plastic, rubber, ceramic, glass, or the like, or a pressure-sensitive member, e.g., a membrane in which fluid permeability varies positively with pressure. Embodiments in which barrier 402 is a frangible member may contain any mechanical or electro-mechanical device known in the art to be suitable for rupturing the membrane.

As illustrated in FIG. 10b, one embodiment involves the insertion of a pestle 640 having a pestle shaft 642 of a length sufficient to reach barrier 402. Affixed to pestle shaft 642 is pestle hilt 644 disposed along the shaft at a position that will allow pestle 640 to make contact with barrier 402, but preventing pestle 642 from contacting chromatography medium 394 non-covalently bound to a protein because of contact made by pestle hilt 644 against inlet port 388. In embodiments in which inlet port 388 is an aperture, the diameter of pestle shaft 642 is less than the diameter of the inlet aperture; in embodiments where inlet port 388 is a valve, the diameter of pestle shaft 642 must be sized to fit through the valve in an open condition. Facilitating barrier disruption is pestle projection 646, which may be thin or thick, one or a plurality, and any of a variety of shapes compatible with rupture or breakage of barrier 402 upon insertion of pestle 640. Other suitable structures to break or rupture barrier 402 include a valve, such as an electrical, mechanical, electro-mechanical, magnetic or electromagnetic valve, a magnetically responsive strike arm pivoted from inner wall surface 392 of second chamber 384, a similarly situated strike arm weakly attached to inner wall surface 392 such that a tap on external wall surface 386 will release the strike arm to make contact with, and break or rupture, barrier 402, and the like.

In addition, barrier 402 is connected to an inner wall surface 392 of infusion module 380 in a manner compatible with formation of a fluid barrier. Exemplary connections are formed by adhering a circumferential face 404 of barrier 402 to a barrier-adhering region 406 of inner wall surface 392 of infusion module 380. Adhesion may be achieved using any technique known in the art, including use of a biocompatible adhesive applied to barrier-adhering region 406 and/or circumferential face 404, heat-mediated localized fusion of circumferential face 404 to barrier-adhering region 406, conformation of circumferential face 404 to barrier-adhering region 406 upon press-fitting barrier 402 to infusion module 380, and the like.

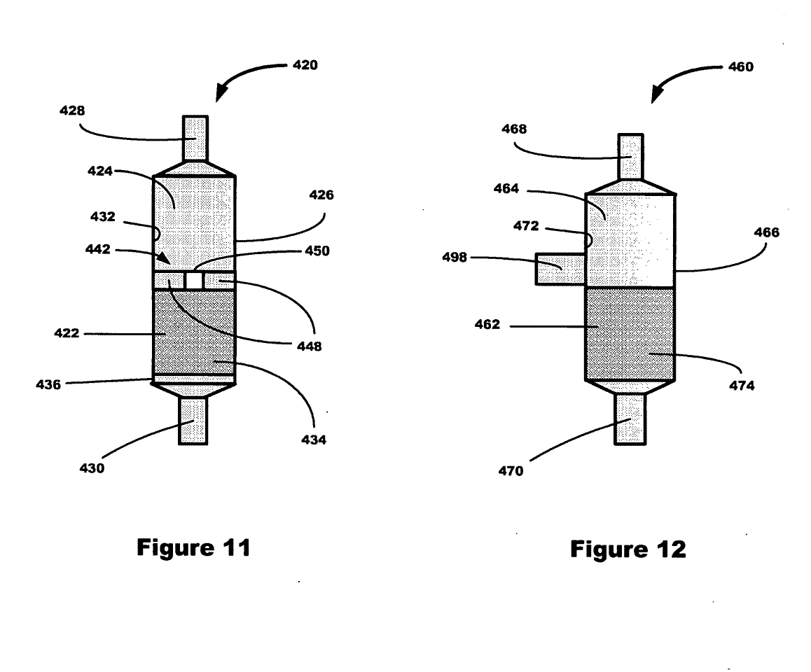

Another embodiment of the infusion module according to the disclosure is provided in FIG. 11, which shows infusion module 420 having an outer wall surface 426, a first chamber 422 containing a chromatography medium 434 non-covalently bound to a protein therapeutic, a second chamber 424, an inlet port 428, an outlet port 430, an outlet filter 436, and a barrier 442. First chamber 422 is defined by outlet filter 436, inner wall surface 432, and barrier 442, while second chamber 424 is defined by barrier 442, inner wall surface 432, and inlet port 428. As illustrated in FIG. 11, barrier 442 has a base member 448 and at least one pre-channel 450, defined as a region of barrier 442 structured to become a preferential channel for fluid flow, for example by being thinner and thus more prone to loss of barrier integrity than base material 448, by being made of a different material than base material 448, wherein the difference makes it easier to form a patent fluid channel through pre-channel 450 than through base material 448, by being geometrically structured to facilitate barrier breach upon an actuating event, such as by focusing the force accompanying increased fluid pressure, insertion and depression of a pestle, and the like.

Still another embodiment of the infusion module according to the disclosure is shown in FIG. 12, wherein infusion module 460 is shown to have an outer wall surface 466, a first chamber 462 containing a chromatography medium 474 non-covalently bound to a protein therapeutic, a second chamber 464, an inlet port 468, an outlet port 470, and an auxiliary input port 498. FIG. 12 illustrates that a fluid, such as an elution fluid, may be introduced via auxiliary input port 498 into a fluid flow passing from inlet port 468 through infusion module 460 and out outlet port 470.

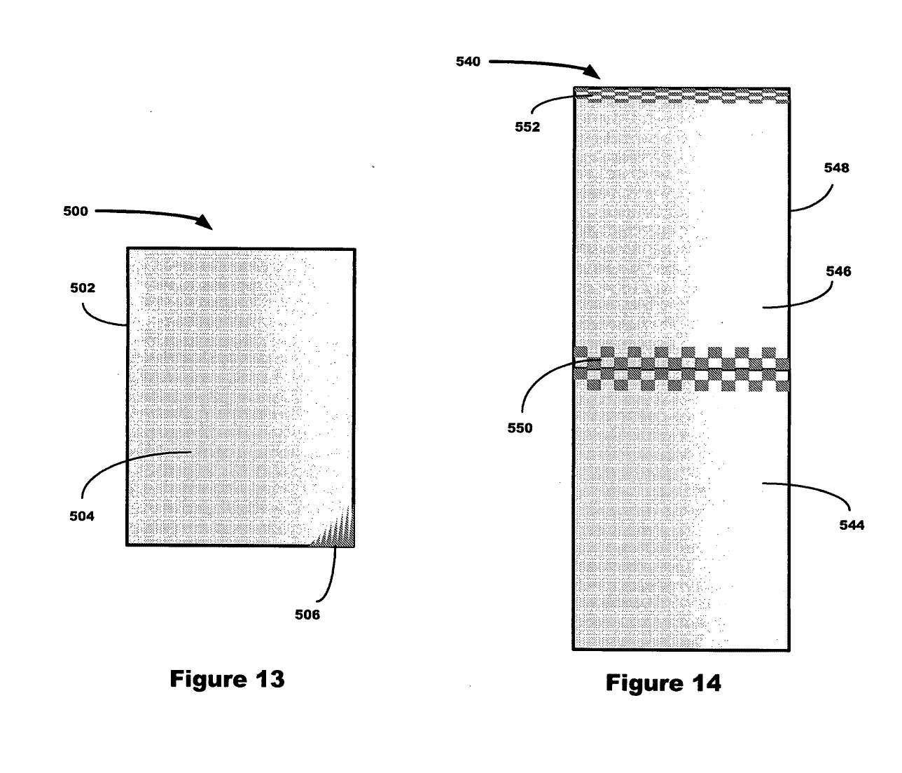

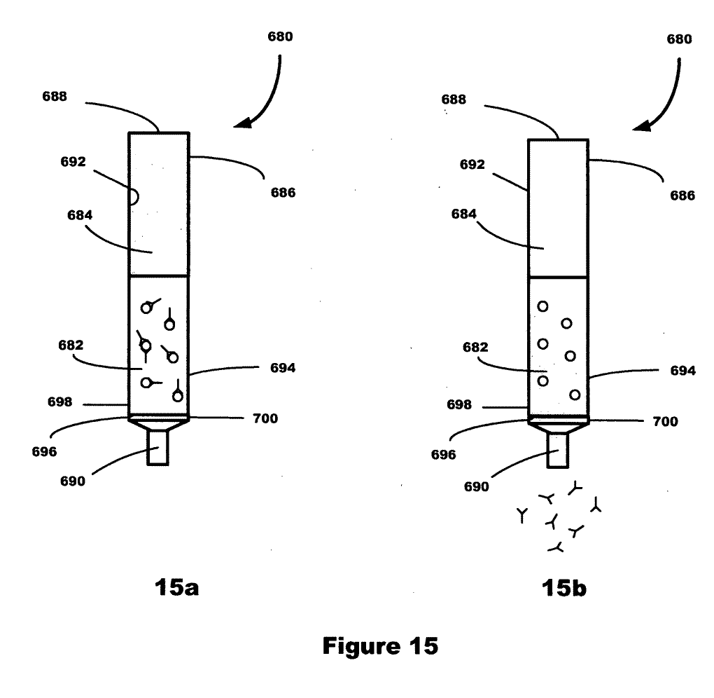

FIG. 13 illustrates an embodiment of another aspect of the disclosure, i.e., a frangible packet 500 having a sealed perimeter 502 defining a packet interior 504 containing a chromatography medium non-covalently bound to a protein, such as a protein therapeutic. As illustrated in FIG. 13, there may be a region 506 of sealed perimeter 502 that is more easily ruptured than the remainder of sealed perimeter 502, thereby tending to direct pressure-induced breakage or rupture of packet 500 to region 506. For ease of illustration, packet 500 is shown as a rectilinear form in plan view, but packet 500 may have any form compatible with a mode of administering a protein, e.g., protein therapeutic, such as use in a generally cylindrical syringe as described herein. Thus, region 506 may be anywhere along the surface of packet 500, such as at an edge or in the field of one or more faces of a particular form used for packet 500, and a packet may or may not contain at least one sealed perimeter 502.

Another embodiment of a packet according to the disclosure is shown in FIG. 14. The packet 540 has exterior sealed perimeter 548 and interior seal 550. Seal 550 is disposed between, and thereby defines, first chamber 544 and second chamber 546. A region 552 of sealed perimeter 548 that is more easily ruptured also may be present in this embodiment of the disclosure. The resistances of inter-chamber seal 550 and external seal 552 to increased fluid pressure typically will, but need not, vary. In certain embodiments, inter-chamber seal 550 exhibits less resistance to increased fluid pressure than external seal 552. In use, e.g., by placement of packet 540 in a syringe according to the disclosure, insertion and actuation of a syringe plunger will increase elution fluid pressure and eventually lead to loss of seal integrity.

Embodiments in which inter-chamber seal 550 is designed to lose its integrity prior to external seal 552, provide an opportunity for the contents of the two chambers to mix before release outside the packet. In one of the chambers, a chromatography medium non-covalently bound to a protein therapeutic is located and in the other chamber is an elution fluid. Actuation of a syringe plunger will bring plunger head 602 (see FIG. 7) into contact with packet 540, thereby increasing the pressure of an elution fluid contained in one of the chambers. Eventually, inter-chamber seal 550 loses its capacity to prevent fluid flow and the elution fluid contacts the chromatography medium, thereby eluting the bound protein. Eventually, packet integrity will be compromised and the eluted protein will be released for delivery via the delivery vehicle, e.g., a syringe.

One of skill will recognize that packaging a chromatography medium non-covalently bound to a protein, such as in the embodiments of the packet illustrated in FIGS. 13 and 14, would allow for bulk preparation and sterilization of the chromatography medium bound to a protein therapeutic, realizing a cost savings. Analogously, packaging the elution fluid in a chamber such as illustrated in FIG. 14 will allow bulk preparation and sterilization of this material.

The embodiments of plunger head 602 shown in FIG. 7 are expected to find use with packets according to the disclosure. The plunger head embodiments of FIG. 7 contain at least one pin (see FIG. 7a), of suitable length, or at least one sharpened point or other shape (see FIG. 7b) suitable for piercing, cutting, scoring or otherwise compromising the structural integrity of a barrier according to the disclosure in a manner such that the compromised barrier exhibits a diminished or lost barrier function. In certain embodiments, plunger head 602 will have a projection in the form of at least one pin, sharpened point, or the like, of sufficient length to make barrier contact before sufficient fluid pressure has developed to compromise the barrier, thereby providing a general alternative to the use of fluid pressure to compromise frangible barriers according to the disclosure. In addition to variable numbers of projections, plunger heads according to the disclosure may have thick or thin projections, long or short projections, and any of a variety of overall shapes compatible with scoring, cutting, puncturing or otherwise compromising the barrier function of a barrier according to the disclosure. Regardless of projection design or length, more than one such projection may be found on plunger head 602, as illustrated in FIG. 7c-d.

The disclosure also provides a system for storing a protein, such as a therapeutic protein, in a stable form. The system comprises a delivery vehicle, such as a delivery vehicle as described above, having at least one chamber containing a chromatography medium non-covalently bound to a protein. The system further comprises an elution fluid calibrated to release at least a portion of the non-covalently bound protein from the chromatography medium. In embodiments in which the bound protein is a therapeutic, the elution fluid is calibrated to release at least one therapeutically effective dose of the protein. The system may be packaged into a kit form, such as a therapeutic kit for treatment or prevention of a disease, disorder or condition amenable to treatment or prevention with a protein therapeutic. The delivery vehicle and elution fluid may be commercially marketed and/or sold together or separately.

### Methods

The methods of administering a protein therapeutic disclosed herein comprehend any form of delivery known in the art that is compatible with elution of a protein therapeutic from the chromatography medium and selective delivery of the therapeutic without delivering the ion exchange medium. Preferred forms for delivery are syringes, including dual-chamber syringes such as the Vetter Lyo-Ject® syringe. In some embodiments, syringes comprise a filter, e.g., an in-line filter, such as a membrane, having a pore size or range of pore sizes that effectively prevents expulsion of the ion exchange medium from the syringe, while allowing expulsion of fluid containing the protein therapeutic. An advantage of using a filter, such as an in-line filter for use in a syringe or for use in intravenous administration, is the capacity to filter any particulate contaminants. Suitable filters include, but are not limited to, a 0.2 μm Gelman Acro sterilizing filter, a Millipak filter, preferably Millipak 100 (Millipore, The Boulevard, Blackmore Lane, Warford, Herts), and the like. In some embodiments, the ion exchange medium is affixed within or upon the filter. Also contemplated is an ion exchange medium sized such that the average diameter of a unit (e.g., bead) of the ion exchange medium exceeds the diameter of the needle aperture, which may be used in a syringe with or without a filter. In related embodiments, the ion exchange medium is chemically cross-linked into fluid-porous forms too large to exit the syringe, with the cross-linking occurring either before or after the packing of the material into the syringe.

A method of administering the immobilized protein is also provided. Administration is accomplished by contacting the immobilized protein in a pre-filled delivery vehicle with an elution fluid such as an elution buffer. Typically, a set volume of elution fluid having a particular pH and/or ionic strength will be used to achieve reliable desorption of a particular dose or quantity of the protein. Flexibility in the choice of elution fluid (and fluid filling the void volume of a pre-filled delivery vehicle) is achieved by keeping eluted volumes small relative to the recipient's blood or other fluid volume or tissue mass, as appropriate depending on the route of administration being used.

In embodiments involving the delivery of a protein therapeutic, the methods according to the disclosure are designed to desorb sufficient protein therapeutic to provide for an effective therapeutic dose notwithstanding the void volume of elution fluid retained in a delivery vehicle such as a pre-filled syringe. In other words, the methods of administration include a sufficient volume of elution fluid of a particular pH and/or ionic strength to elute an effective therapeutic dose in that portion of the elution fluid that is delivered or administered, rather than being retained in the void volume of a delivery vehicle containing an ion exchange medium. In embodiments involving continuous or semi-continuous delivery of a protein therapeutic, such as when using an in-line pre-filled vehicle in an intravenous delivery system, considerations of protein therapeutic loss in a void volume will not apply. Rather, in such situations, the characteristics of the elution fluid, e.g., the pH and/or ionic strength, will be set at levels designed to promote the steady desorption of an effective dose of protein therapeutic over time.

While it may be possible to administer a compound alone, in the methods described, the compound administered is generally present as an active ingredient in a desired dosage unit formulation, such as a pharmaceutically acceptable composition containing a conventional pharmaceutically acceptable carrier. Thus, in another aspect of the disclosure, there is provided a pharmaceutical composition comprising a therapeutic compound in combination with a pharmaceutically acceptable carrier. Acceptable pharmaceutical carriers generally include diluents, excipients, adjuvants and the like, as described herein.

A pharmaceutical composition of the disclosure may comprise an effective amount of a protein therapeutic or an effective dosage amount of a protein therapeutic. An effective dosage amount of a compound includes an amount less than, equal to, or greater than an effective amount of the compound. For example, a pharmaceutical composition in which two or more unit dosages, such as in tablets, capsules and the like, are required to administer an effective amount of the compound, or alternatively, a multi-dose pharmaceutical composition, such as powders, liquids and the like, in which an effective amount of the compound may be administered by administering a portion of the composition. The compositions also may provide for the delivery of concentrated dosages of protein therapeutics up to 300 mg/ml. The concentration, and/or viscosity, of the administered therapeutic are amenable to control by adjusting the volume of elution fluid.

More generally, an immobilized protein according to the disclosure may be formulated in a tablet, capsule, powder or any other pharmaceutical formulation known in the art for convenient use in the delivery vehicle (e.g., a syringe or infusion module). Further, the immobilized protein formulations may be packaged, e.g., as sterile or non-sterile formulations in the packet described herein and illustrated in FIGS. 12 and 13.

The pharmaceutical compositions may generally be prepared by mixing one or more protein compounds with one or more pharmaceutically acceptable carriers, excipients, binders, adjuvants, diluents, preservatives, solubilizers, emulsifiers and the like, to form a desired administrable formulation to treat, ameliorate or prevent a variety of diseases. Such compositions include diluents of various buffer content (e.g., Tris-HCl, acetate, phosphate), pH and ionic strength; additives such as detergents and solubilizing agents (e.g., Tween 80, Polysorbate 80), anti-oxidants (e.g., ascorbic acid, sodium metabisulfite), preservatives (e.g., thimerasol, benzyl alcohol) and bulking substances (e.g., lactose, mannitol); incorporation of the material into particulate preparations of polymeric compounds such as polylactic acid, polyglycolic acid, etc. or into liposomes. Hyaluronic acid may also be used, and this may have the effect of promoting sustained duration in the circulation. Such compositions may influence the physical state, stability, rate of in vivo release, and rate of in vivo clearance of the present proteins and derivatives. See, e.g., Remington's Pharmaceutical Sciences, 18th Ed. (1990, Mack Publishing Co., Easton, Pa. 18042) pages 1435-1712 which are herein incorporated by reference.

The pharmaceutical compositions may be subjected to conventional pharmaceutical operations such as sterilization and/or may contain conventional adjuvants, such as preservatives, stabilizers, wetting agents, emulsifiers, buffers etc. The pharmaceutically active compounds of this disclosure can be processed in accordance with conventional methods of pharmacy to produce medicinal agents for administration to patients, including humans and other mammals.

Pharmaceutical compositions can be can be administered in a local rather than a systemic fashion, such as injection as a sustained release formulation.

Besides those representative dosage forms described herein, pharmaceutically acceptable excipients and carriers are generally known to those skilled in the art and are thus contemplated. Such excipients and carriers are described, for example, in “Remingtons Pharmaceutical Sciences” Mack Pub. Co., New Jersey (2000); and “Pharmaceutics The Science of Dosage Form Design, 2^{nd} Ed. (Aulton, ed.) Churchill Livingstone (2002). The following dosage forms are given by way of example and should not be construed as limiting.

Protein therapeutics according to the disclosure are typically administered by injection, including but not limited to, parenteral, intravenous, intramuscular, subcutaneous and intraperitoneal injection.

Injectable dosage forms for parenteral administration generally include aqueous suspensions or oil suspensions, which may be prepared using a suitable dispersant or wetting agent and a suspending agent. Injectable forms may be in solution phase or a powder suitable for reconstitution as a solution. Both are prepared with a solvent or diluent. Acceptable solvents or vehicles include sterilized water, Ringer's solution, or an isotonic aqueous saline solution. Alternatively, sterile oils may be employed as solvents or suspending agents. Typically, the oil or fatty acid is non-volatile, including natural or synthetic oils, fatty acids, mono-, di- or tri-glycerides. For injection, the formulations may optionally contain stabilizers, pH modifiers, surfactants, bioavailability modifiers and combinations of these. The compounds may be formulated for parenteral administration by injection such as by bolus injection or continuous infusion. A unit dosage form for injection may be in delivery vehicles in the form of ampoules or in multi-dose delivery vehicles, e.g., multi-dose infusion modules.

Specific dosages may be adjusted depending on conditions of disease, the age, body weight, general health conditions, sex, and diet of the subject, dose intervals, administration routes, excretion rate, and combinations of drugs. Any of the above dosage forms containing effective amounts are well within the bounds of routine experimentation and therefore, well within the scope of the instant disclosure.

A therapeutically effective dose may vary depending upon the route of administration and dosage form. Typically, the compound or compounds as disclosed herein are selected to provide a formulation that exhibits a high therapeutic index. The therapeutic index is the dose ratio between toxic and therapeutic effects which can be expressed as the ratio between LD₅₀ and ED₅₀. The LD₅₀ is the dose lethal to 50% of the population and the ED₅₀ is the dose therapeutically effective in 50% of the population. The LD₅₀ and ED₅₀ are determined by standard pharmaceutical procedures in animal cell cultures or experimental animals.

A dosage regimen for treating a diseases or disorder is based on a variety of factors, including the type of disease, the age, weight, sex, medical condition of the patient, the severity of the condition, the route of administration, and the particular compound employed. Thus, the dosage regimen may vary widely, but can be determined routinely using standard methods. Dosage levels of the order from about 0.01 mg to 30 mg per kilogram of body weight per day, for example from about 0.1 mg to 10 mg/kg, or from about 0.25 mg to 1 mg/kg are useful for all methods of use disclosed herein. Generally, the daily regimen should be in the range of 0.1-1000 micrograms of the compound per kilogram of body weight, preferably 0.1-150 micrograms per kilogram.

The active ingredient may also be administered by injection or infusion as a composition, optionally with suitable carriers including saline, dextrose, or water. The daily parenteral dosage regimen will be from about 0.1 to about 30 mg/kg of total body weight, such as from about 0.1 to about 10 mg/kg, or from about 0.25 mg to 1 mg/kg.

The disclosure further provides a method of producing the stable pre-filled delivery vehicles. Generally the method involves the application of a protein contained in a loading buffer to chromatography medium, such as an ion exchange medium, under conditions of pH and ionic strength permissive for non-covalent binding of the protein therapeutic to the ion exchange medium either before or after the medium is added to a delivery vehicle. The choice of delivery vehicle, chromatography medium (e.g., ion exchange medium), binding capacity thereof, loading buffer pH, loading buffer ionic strength and protein concentration in the loading buffer are recognized by those of skill in the art as varying depending on the particular circumstances and methods of production will be adapted to accommodate such circumstances. It is preferred that the method of production further comprise a washing step to eliminate unbound protein and contaminants. Following production, the protein in stable form, i.e., in the form of pre-filled delivery vehicles, is stored for days, weeks, months, or longer, typically at room temperature or under refrigeration. The stability of the formulations, however, permit storage for considerable time periods in the field at ambient temperatures.

### Kits

The disclosure also provides kits for stable storage and administration of a therapeutic comprising a pre-filled delivery vehicle and instruction for use thereof. A pre-filled delivery vehicle is any vehicle for delivering a protein, such as a protein therapeutic, that is capable of selectively delivering a desorbed protein without concomitant delivery of a chromatography medium, whether that capacity arises from separation of the desorbed protein and the medium or retention of the medium in the vehicle. An exemplary delivery vehicle is a pre-filled syringe or an infusion module in fluid communication with an intravenous administration system, such as an infusion module in-line with intravenous administration tubing. The instruction for use may be a package insert and will provide guidance on the use of the delivery vehicle in delivering, or administering, at least one dose of a protein, e.g., a protein therapeutic.

### Components

Proteins suitable for use in the delivery vehicles, systems, methods, and kits according to the disclosure include any protein or fragment, derivative or variant thereof, that is known in the art. Such proteins include a wide variety of monomeric, homo-multimeric and hetero-multimeric holo-proteins, as well as single-chain subunits, fragments, derivatives, and peptides. Some of these proteins will have a known therapeutic use, such as peptide hormones, peptide ligands, signaling molecules (e.g., cytokines, chemokines), and antibodies. Any form of therapeutically active protein, e.g., any form of a therapeutically active antibody (e.g., monoclonal or polyclonal, intact antibody or fragment thereof (Fab, F(ab′)₂,) obtained from any animal or antibody-producing cell source, such as a mammal or mammalian cell, chimeric, humanized, and human antibodies of any isotype or mixed isotype, single-chain molecules including scFv, diabody, recombinant antibody forms, and camelid antibodies, and the like.

Non-limiting examples of proteins suitable for use according to the disclosure include a protein, such as a therapeutic protein, that is selected from the group consisting of etanercept (Enbrel®, an anti-TNFα antibody), erythropoietin, darbepoetin alfa (Aranesp®, an EPO analog), filgrastim (Neupogen® or recombinant methionyl human granulocyte colony-stimulating factor (r-metHuG-CSF)) and pegfilgrastim (Neulasta®, a PEGylated filgrastim). Embodiments of the protein therapeutic also include therapeutic antibodies such as Humira (adalimumab), Synagis (palivizumab), 146B7-CHO, vectibix (panitumumab), Rituxan (rituximab), zevalin (ibritumomab tiuxetan), anti-CD80 monoclonal antibody (mAb) (galiximab), anti-CD23 mAb (lumiliximab), M200 (volociximab), anti-Cripto mAb, anti-BR3 mAb, anti-IGF1R mAb, Tysabri (natalizumab), Daclizumab, humanized anti-CD20 mAb (ocrelizumab), soluble BAFF antagonist (BR3-Fc), anti-CD40L mAb, anti-TWEAK mAb, anti-IL5 Receptor mAb, anti-ganglioside GM2 mAb, anti-FGF8 mAb, anti-VEGFR/Flt-1 mAb, anti-ganglioside GD2 mAb, Actilyse® (alteplase), Metalyse® (tenecteplase), CAT-3888 and CAT-8015 (anti-CD22 dsFv-PE38 conjugates), CAT-354 (anti-IL13 mAb), CAT-5001 (anti-mesothelin dsFv-PE38 conjugate), GC-1008 (anti-TGF-β mAb), CAM-3001 (anti-GM-CSF Receptor mAb), ABT-874 (anti-IL12 mAb), Lymphostat B (Belimumab; anti-BlyS mAb), HGS-ETR1 (mapatumumab; human anti-TRAIL Receptor-1 mAb), HGS-ETR2 (human anti-TRAIL Receptor-2 mAb), ABthrax™ (human, anti-protective antigen (from B. anthracis) mAb), MYO-029 (human anti-GDF-8 mAb), CAT-213 (anti-eotaxin1 mAb), Erbitux, Epratuzumab, Remicade (infliximab; anti-TNF mAb), Herpceptin (traztusumab), ReoPro (abciximab), Actemra (anti-IL6 Receptor mAb), Avastin, HuMax-CD4 (zanolimumab), HuMax-CD20 (ofatumumab), HuMax-EGFr (zalutumumab), HuMax-Inflam, 81507 (anti-IGF-1R mAb), HuMax HepC, HuMax CD38, HuMax-TAC (anti-IL2Ra or anti-CD25 mAb), HuMax-ZP3 (anti-ZP3 mAb), Bexxar (tositumomab), Orthoclone OKT3 (muromonab-CD3), MDX-010 (ipilimumab), anti-CTLA4, CNTO 148 (golimumab; anti-TNFα Inflammation mAb), CNTO 1275 (anti-IL12/IL23 mAb), HuMax-CD4 (zanolimumab), HuMax-CD20 (ofatumumab), HuMax-EGFR (zalutumumab), MDX-066 (CDA-1) and MDX-1388 (anti-C. difficile Toxin A and Toxin B C mAbs), MDX-060 (anti-CD30 mAb), MDX-018, CNTO 95 (anti-integrin receptors mAb), MDX-1307 (anti-Mannose Receptor/hCGβ mAb), MDX-1100 (anti-1P10 Ulcerative Colitis mAb), MDX-1303 (Valortim™), anti-B. anthracis Anthrax, MEDI-545 (MDX-1103, anti-IFNa), MDX-1106 (ONO-4538; anti-PD1), NVS Antibody #1, NVS Antibody #2, FG-3019 (anti-CTGF Idiopathic Pulmonary Fibrosis Phase I Fibrogen), LLY Antibody, BMS-66513, NI-0401 (anti-CD3 mAb), IMC-18F1 (VEGFR-1), IMC-3G3 (anti-PDGFRα), MDX-1401 (anti-CD30), MDX-1333 (anti-IFNAR), Synagis (palivizumab; anti-RSV mAb), Campath (alemtuzumab), Velcade (bortezomib), MLN0002 (anti-alpha4beta7 mAb), MLN1202 (anti-CCR2 chemokine receptor mAb)., Simulect (basiliximab), prexige (lumiracoxib), Xolair (omalizumab), ETI211 (anti-MRSA mAb), IL-1 Trap (the Fc portion of human IgG1 and the extracellular domains of both IL-1 receptor components (the Type I receptor and receptor accessory protein)), VEGF Trap (Ig domains of VEGFR1 fused to IgG1 Fc), Zenapax (Daclizumab), Avastin (Bevacizumab), MabThera (Rituximab), MabTheraRA (Rituximab), Tarceva (Erlotinib), Zevalin (ibritumomab tiuxetan), Zetia (ezetimibe), Zyttorin (ezetimibe and simvastatin), Atacicept (TACI-Ig), NI-0401 (anti-CD3 in Crohn's disease), Adecatumumab, Golimumab (anti-TNFα mAb), Epratuzumab, Gemtuzumab, Raptiva (efalizumab), Cimzia (certolizumab pegol, CDP 870), (Soliris) Eculizumab, Pexelizumab (Anti-C5 Complement), MEDI-524 (Numax), Lucentis (Ranibizumab), 17-1A (Panorex), Trabio (lerdelimumab), TheraCim hR3 (Nimotuzumab), Omnitarg (Pertuzumab), Osidem (IDM-1), OvaRex (B43.13), Nuvion (visilizumab), and Cantuzamab. Other embodiments of the disclosure comprise a protein therapeutic that is not an antibody, such as a peptide hormone, a peptide ligand, signaling molecules such as cytokines and chemokines, or any protein known to exert a therapeutically beneficial effect, such as natrecor (nesiritide; rh type B natriuretic peptide) erythropoietin (see above), insulin, Insulin in Solution, INFERGEN® (Interferon alfacon-1), KINERET® (anakinra), Mylotarg (gemtuzumab ozogamicin), ROFERON®-A (Interferon alfa-2a), VECTIBLIX (panatumamab), and the like. Also contemplated are fusion proteins such as peptibodies, avimers, and fragments, derivatives and variants thereof. In certain embodiments, the protein therapeutic has a pI of at least 7.0.

Among particular illustrative proteins are certain antibody and antibody-related proteins, including Fc fusion protein and peptibodies, such as, for instance, those listed immediately below and elsewhere herein and other fusion proteins comprising an Fc region or a fragment or derivative thereof:

OPGL-specific antibodies, peptibodies, and related proteins, and the like (also referred to as RANKL specific antibodies, peptibodies and the like), including fully humanized and human OPGL specific antibodies, particularly fully humanized monoclonal antibodies, including but not limited to, the antibodies described in International (PCT) Patent Application Publication Number WO 03/002713, which is incorporated herein by reference in its entirety as to OPGL-specific antibodies and antibody related proteins, particularly those having the sequences set forth therein, particularly, but not limited to, those denoted therein, i.e., 9H7; 18B2; 2D8; 2E11; 16E1; and 22B3, including the OPGL-specific antibodies having either the light chain of SEQ ID NO: 2 as set forth therein in FIG. 2 and/or the heavy chain of SEQ ID NO:4, as set forth therein in FIG. 4, each of which is individually and specifically incorporated by reference herein in its entirety.

Myostatin-binding proteins, peptibodies, related proteins, and the like, including myostatin-specific peptibodies, particularly those described in US Patent Application Publication Number 2004/0181033 and International (PCT) Patent Application Publication Number WO2004/058988 which are each incorporated by reference herein in its entirety, particularly in parts pertinent to myostatin-specific peptibodies, including but not limited to peptibodies of the mTN8-19 family, including those of SEQ ID NOS: 305-351 therein, including TN8-19-1 through TN8-19-40, TN8-19 coni and TN8-19 cont; peptibodies of the mL2 family of SEQ ID NOS: 357-383 therein; the mL15 family of SEQ ID NOS: 384-409 therein; the mL17 family of SEQ ID NOS: 410-438 therein; the mL20 family of SEQ ID NOS: 439-446 therein; the mL21 family of SEQ ID NOS: 447-452 therein; the mL24 family of SEQ ID NOS: 453-454 therein; and those of SEQ ID NOS: 615-631 therein, each of which is individually and specifically incorporated by reference herein in its entirety.

IL-4 receptor-specific antibodies, peptibodies, and related proteins, and the like, particularly those that inhibit activities mediated by binding of IL-4 and/or IL-13 to the receptor, including those described in International (PCT) Patent Application Publication No. WO 2005/047331 of International (PCT) Patent Application Number PCT/US2004/03742 and in US Patent Application Publication Number 2005/112694, each of which is incorporated herein by reference in its entirety, particularly in parts pertinent to IL-4 receptor-specific antibodies, particularly such antibodies as are described therein, particularly, and without limitation, those designated therein, i.e., L1H1; L1H2; L1H3; L1H4; L1H5; L1H6; L1H7; L1H8; L1H9; L1H10; L1H11; L2H1; L2H2; L2H3; L2H4; L2H5; L2H6; L2H7; L2H8; L2H9; L2H10; L2H11; L2H12; L2H13; L2H14; L3H1; L4H1; L5H1; L6H1, each of which is individually and specifically incorporated by reference herein in its entirety.

Interleukin 1-receptor 1 (“IL1-R1”) specific antibodies, peptibodies, related proteins, and the like, including but not limited to those described in U.S. Patent Application Publication Number US2004/097712A1, which is incorporated herein by reference in its entirety in parts pertinent to IL1-R1 specific binding proteins, monoclonal antibodies in particular, especially, without limitation, those designated therein, i.e., 15CA, 26F5, 27F2, 24E12, and 10H7, each of which is individually and specifically incorporated by reference herein in its entirety.

Ang2-specific antibodies, peptibodies, related proteins, and the like, including but not limited to those described in International (PCT) Patent Application Publication Number WO 03/057134 and U.S. Patent Application Publication Number US2003/0229023, each of which is incorporated herein by reference in its entirety, particularly in parts pertinent to Ang2-specific antibodies and peptibodies and the like, especially those of sequences described therein and including but not limited to, L1(N); L1(N) WT; L1(N) 1K WT; 2xL1(N); 2xL1(N) WT; Con4 (N), Con4 (N) 1K WT, 2xCon4 (N) 1K; L1©; L1© 1K; 2xL1©; Con4©; Con4© 1K; 2xCon4© 1K; Con4-L1 (N); Con4-L1©; TN-12-9 (N); C17 (N); TN8-8(N); TN8-14 (N); Con 1 (N), also including anti-Ang 2 antibodies and formulations, such as those described in International (PCT) Patent Application Publication Number WO 2003/030833, which is incorporated herein by reference in its entirety as to the same, particularly Ab526; Ab528; Ab531; Ab533; Ab535; Ab536; Ab537; Ab540; Ab543; Ab544; Ab545; Ab546; A551; Ab553; Ab555; Ab558; Ab559; Ab565; AbF1AbFD; AbFE; AbFJ; AbFK; AbGID4; AbGC1E8; AbH1C12; AblA1; AblF; AblK, AblP; and AblP, in their various permutations as described therein, each of which is individually and specifically incorporated by reference herein in its entirety.

NGF-specific antibodies, peptibodies, related proteins, and the like, including, but not limited to, those proteins described in U.S. Patent Application Publication Number US2005/0074821 and U.S. Pat. No. 6,919,426, each of which is incorporated herein by reference in its entirety, particularly as to NGF-specific antibodies and related proteins, including but not limited to, the NGF-specific antibodies therein designated as 4D4, 4G6, 6H9, 7H2, 14D10 and 14D11, each of which is individually and specifically incorporated by reference herein in its entirety.

CD22-specific antibodies, peptibodies, related proteins, and the like, such as those described in U.S. Pat. No. 5,789,554, which is incorporated herein by reference in its entirety as to CD22-specific antibodies and related proteins, particularly human CD22-specific antibodies such as, but not limited to, humanized and fully human antibodies, including but not limited to, humanized and fully human monoclonal antibodies, particularly including but not limited to, human CD22-specific IgG antibodies, such as, for instance, a dimer of a human-mouse monoclonal hLL2 gamma-chain disulfide linked to a human-mouse monoclonal hLL2 kappa-chain, including, but limited to, e.g., the human CD22-specific fully humanized antibody in Epratuzumab, CAS registry number 501423-23-0.

IGF-1 receptor-specific antibodies, peptibodies, related proteins, and the like, such as those described in International (PCT) Patent Application Number PCT/US2005/046493, which is incorporated herein by reference in its entirety as to IGF-1 receptor-specific antibodies and related proteins, including but not limited to the IGF-1 specific antibodies therein designated L1H1, L2H2, L3H3, L4H4, L5H5, L6H6, L7H7, L8H8, L9H9, L10H10, L11H11, L12H12, L13H13, L14H14, L15H15, L16H16, L17H17, L18H18, L19H19, L20H20, L21H21, L22H22, L23H23, L24H24, L25H25, L26H26, L27H27, L28H28, L29H29, L30H30, L31H31, L32H32, L33H33, L34H34, L35H35, L36H36, L37H37, L38H38, L39H39, L40H40, L41H41, L42H42, L43H43, L44H44, L45H45, L46H46, L47H47, L48H48, L49H49, L50H50, L51H51, L52H52, and IGF-1R-binding fragments and derivatives thereof, each of which is individually and specifically incorporated by reference herein in its entirety.

Also among non-limiting examples of anti-IGF-IR antibodies for use in the methods and compositions of the present invention are each and all of those described in at least one of the following publications:

U.S. Patent Application Publication Numbers 06/0040358 (published Feb. 23, 2006), 05/0008642 (published Jan. 13, 2005), 04/0228859 (published Nov. 18, 2004), including but not limited to, for instance, antibody 1A (DSMZ Deposit No. DSM ACC 2586), antibody 8 (DSMZ Deposit No. DSM ACC 2589), antibody 23 (DSMZ Deposit No. DSM ACC 2588) and antibody 18, as described therein;

International (PCT) Patent Application Publication Numbers WO 06/138729 (published Dec. 28, 2006), WO 05/016970 (published Feb. 24, 2005), and Lu et al., 2004, J Biol Chem. 279:2856-65, including but not limited to antibodies 2F8, A12, and IMC-A12, as described therein;

International (PCT) Patent Application Publication Numbers WO 07/012,614 (published Feb. 1, 2007), WO 07/000,328 (published Jan. 4, 2007), WO 06/013472 (published Feb. 9, 2006), WO 05/058967 (published Jun. 30, 2005), and WO 03/059951 (published Jul. 24, 2003);

U.S. Patent Application Publication Number 05/0084906 (published Apr. 21, 2005), including but not limited to antibody 7C10, chimeric antibody C7C10, antibody h7C10, antibody 7H2M, chimeric antibody *7C10, antibody GM 607, humanized antibody 7C10 version 1, humanized antibody 7C10 version 2, humanized antibody 7C10 version 3, and antibody 7H2HM, as described therein;

U.S. Patent Application Publication Numbers 05/0249728 (published Nov. 10, 2005), 05/0186203 (published Aug. 25, 2005), 04/0265307 (published Dec. 30, 2004), and 03/0235582 (published Dec. 25, 2003) as well as Maloney et al., 2003, Cancer Res. 63:5073-83, including but not limited to antibody EM164, resurfaced EM164, humanized EM164, huEM164 v1.0, huEM164 v1.1, huEM164 v1.2, and huEM164 v1.3, as described therein;

U.S. Pat. No. 7,037,498 (issued May 2, 2006), U.S. Patent Application Publication Numbers 05/0244408 (published Nov. 30, 2005), and 04/0086503 (published May 6, 2004), as well as Cohen, et al., 2005, Clinical Cancer Res. 11:2063-73, e.g., antibody CP-751,871, including but not limited to each of the antibodies produced by the hybridomas having the ATCC accession numbers PTA-2792, PTA-2788, PTA-2790, PTA-2791, PTA-2789, PTA-2793, and antibodies 2.12.1, 2.13.2, 2.14.3, 3.1.1, 4.9.2, and 4.17.3, as described therein;

U.S. Patent Application Publication Numbers 05/0136063 (published Jun. 23, 2005), and 04/0018191 (published Jan. 29, 2004), including but not limited to antibody 19D12 and an antibody comprising a heavy chain encoded by a polynucleotide in plasmid 15H12/19D12 HCA (γ4), deposited at the ATCC under accession number PTA-5214, and a light chain encoded by a polynucleotide in plasmid 15H12/19D12 LCF (κ), deposited at the ATCC under accession number PTA-5220, as described therein;

U.S. Patent Application Publication Number 04/0202655 (published Oct. 14, 2004), including but not limited to antibodies PINT-6A1, PINT-7A2, PINT-7A4, PINT-7A5, PINT-7A6, PINT-8A1, PINT-9A2, PINT-11A1, PINT-11A2, PINT-11A3, PINT-11A4, PINT-11A5, PINT-11A7, PINT-11A12, PINT-12A1, PINT-12A2, PINT-12A3, PINT-12A4, and PINT-12A5, as described therein;

Each and all of the proteins identified above or elsewhere herein are each incorporated by reference in their entireties, including the sequence thereof, particularly as to the aforementioned antibodies, peptibodies, related proteins, and the like that target IGF-1 receptors.

B-7 related protein 1-specific antibodies, peptibodies, related proteins and the like (“B7RP-1,” also is referred to in the literature as B7H2, ICOSL, B7h, and CD275), particularly B7RP-specific fully human monoclonal IgG2 antibodies, particularly fully human IgG2 monoclonal antibody that binds an epitope in the first immunoglobulin-like domain of B7RP-1, especially those that inhibit the interaction of B7RP-1 with its natural receptor, ICOS, on activated T cells, particularly, in all of the foregoing regards, those proteins disclosed in U.S. Provisional Patent Application No. 60/700,265, filed 18 Jul. 2005 and International (PCT) Patent Application Publication Number WO07/011,941, each of which is incorporated herein by reference in its entirety as to such antibodies and related proteins, including but not limited to antibodies designated therein as 16H (having light chain variable and heavy chain variable sequences of SEQ ID NO:1 and SEQ ID NO:7 therein, respectively); 5D (having light chain variable and heavy chain variable sequences of SEQ ID NO:2 and SEQ ID NO:9 therein, respectively); 2H (having light chain variable and heavy chain variable sequences of SEQ ID NO:3 and SEQ ID NO:10 therein, respectively); 43H (having light chain variable and heavy chain variable sequences of SEQ ID NO:6 and SEQ ID NO:14 therein, respectively); 41H (having light chain variable and heavy chain variable sequences of SEQ ID NO:5 and SEQ ID NO:13 therein, respectively); and 15H (having light chain variable and heavy chain variable sequences of SEQ ID NO:4 and SEQ ID NO:12 therein, respectively), each of which is individually and specifically incorporated by reference herein in its entirety.

IL-15-specific antibodies, peptibodies, related proteins, and the like, such as, in particular, humanized monoclonal antibodies, particularly antibodies such as those disclosed in U.S. Patent Application Publication Numbers US2003/0138421, US2003/023586, and US2004/0071702, as well as U.S. Pat. No. 7,153,507, each of which is incorporated herein by reference in its entirety as to IL-15-specific antibodies and related proteins, including peptibodies, and including but not limited to HuMax IL-15 antibodies and related proteins, e.g., 146B7.

Interferon (IFN) gamma-specific antibodies, peptibodies, related proteins and the like, especially human IFN gamma-specific antibodies, particularly fully human anti-IFN gamma antibodies, such as, for instance, those described in U.S. Patent Application Publication Number US2005/0004353, which is incorporated herein by reference in its entirety as to IFN gamma-specific antibodies, particularly, for example, the antibodies therein designated 1118; 1118*; 1119; 1121; and 1121*, each of which is individually and specifically incorporated by reference herein in its entirety.

TALL-1-specific antibodies, peptibodies, related proteins and the like, and other TALL-specific binding proteins, such as those described in U.S. Patent Application Publication Numbers 2003/0195156 and 2006/135431, each of which is incorporated herein by reference in its entirety as to TALL-1 binding proteins, particularly the molecules of Tables 4 and 5B therein, each of which is individually and specifically incorporated by reference herein in its entirety.

Parathyroid hormone (“PTH”)-specific antibodies, peptibodies, related proteins, and the like, such as those described in U.S. Pat. No. 6,756,480, which is incorporated herein by reference in its entirety, particularly in parts pertinent to proteins that bind PTH.

Thrombopoietin receptor (“TPO-R”)-specific antibodies, peptibodies, related proteins, and the like, such as those described in U.S. Pat. No. 6,835,809, which is incorporated herein by reference in its entirety, particularly in parts pertinent to proteins that bind TPO-R.

Hepatocyte growth factor (“HGF”)-specific antibodies, peptibodies, related proteins, and the like, including those that target the HGF/SF:cMet axis (HGF/SF:c-Met), such as the fully human monoclonal antibodies that neutralize hepatocyte growth factor/scatter (HGF/SF) described in U.S. Patent Application Publication Number US2005/0118643 and International (PCT) Patent Application Publication Number WO2005/017107, huL2G7 described in U.S. Pat. No. 7,220,410, and OA-5d5, described in U.S. Pat. Nos. 5,686,292, and 6,468,529, and in International (PCT) Patent Application Publication Number WO 96/38557, each of which is incorporated herein by reference in its entirety, particularly in parts pertinent to proteins that bind HGF.

TRAIL-R2-specific antibodies, peptibodies, related proteins and the like, such as those described in U.S. Provisional Patent Application Nos. 60/713,433, filed 31 Aug. 2005, and 60/713,478, filed 31 Aug. 2005, each of which is incorporated herein by reference in its entirety, particularly in parts pertinent to proteins that bind TRAIL-R2.

Activin A-specific antibodies, peptibodies, related proteins, and the like, including but not limited to those proteins described in U.S. Provisional Patent Application No. 60/843,430, filed Sep. 8, 2006, which is incorporated herein by reference in its entirety, particularly in parts pertinent to proteins that bind Activin A.

TGF-β-specific antibodies, peptibodies, related proteins, and the like, including but not limited to those described in U.S. Pat. No. 6,803,453 and U.S. Patent Application Publication Number 2007/110747, each of which is incorporated herein by reference in its entirety, particularly in parts pertinent to proteins that bind TGF-β.

Amyloid-beta protein-specific antibodies, peptibodies, related proteins, and the like, including but not limited to those proteins described in International (PCT) Patent Application Publication Number WO2006/081171, which is incorporated herein by reference in its entirety, particularly in parts pertinent to proteins that bind amyloid-beta proteins.

Additional exemplary proteins according to the disclosure are antibodies beyond those noted above, and other types of target-binding proteins, as well as proteins relating thereto or derived therefrom, and protein ligands, and proteins derived therefrom or relating thereto, particularly those comprising an Fc region of an antibody or a region derived from an Fc region. Of note among these proteins are ligand-binding proteins that bind signal and/or effector proteins, and proteins relating thereto or derived therefrom.

Among such binding proteins, including Fc fusion proteins, proteins derived therefrom and proteins related thereto, are those that bind to one or more of the following targets, alone or in any combination.

(i) CD proteins including, but not limited to, CD3, CD4, CD8, CD19, CD20, CD22, CD30, and CD34; including those that interfere with receptor binding.

(ii) HER receptor family proteins, including, for example, HER2, HER3, HER4, and the EGF receptor;

(iii) cell adhesion molecules, e.g., LFA-1, Mol, p150,95, VLA-4, ICAM-1, VCAM, and alpha v/beta 3 integrin;

(iv) growth factors, including but not limited to, for example, vascular endothelial growth factor (“VEGF”), growth hormone, thyroid stimulating hormone, follicle stimulating hormone, luteinizing hormone, growth hormone releasing factor, parathyroid hormone, mullerian-inhibiting substance, human macrophage inflammatory protein (MIP-1α), erythropoietin (EPO), nerve growth factor, such as NGF-beta, platelet-derived growth factor (PDGF), fibroblast growth factors, including, for instance, aFGF and bFGF, epidermal growth factor (EGF), transforming growth factors (TGF), including, among others, TGF-α and TGF-β, including TGF-β1, TGF-β2, TGF-β3, TGF-β4, or TGF-β5, insulin-like growth factors-I and -II (IGF-I and IGF-II), des(1-3)-IGF-I (brain IGF-I), and osteoinductive factors;

(v) insulins and insulin-related proteins, including but not limited to insulin, insulin A-chain, insulin B-chain, proinsulin, and insulin-like growth factor binding proteins;

(vi) coagulation and coagulation-related proteins, such as, among others, factor VIII, tissue factor, von Willebrand's factor, protein C, alpha-1-antitrypsin, plasminogen activators, such as urokinase and tissue plasminogen activator (“t-PA”), bombazine, thrombin, and thrombopoietin;

(vii) colony stimulating factors (CSFs) and receptors thereof, including the following, among others, M-CSF, GM-CSF, and G-CSF, and receptors thereof, such as CSF-1 receptor (c-fms);

(viii) other blood and serum proteins, including but not limited to albumin, IgE, and blood group antigens;

(ix) receptors and receptor-associated proteins, including, for example, flk2/flt3 receptor, obesity (OB) receptor, growth hormone receptors, thrombopoietin receptors (“TPO-R,” “c-mpl”), glucagon receptors, interleukin receptors, interferon receptors, T-cell receptors, and other receptors listed herein;

(x) neurotrophic factors, including but not limited to, bone-derived neurotrophic factor (BDNF) and neurotrophin-3, -4, -5, or -6 (NT-3, NT-4, NT-5, or NT-6);

(xi) relaxin A-chain, relaxin B-chain, and prorelaxin;

(xii) interferons and interferon receptors, including, for example, interferonα, -β, and -γ, and interferon-α, -β, and -γ receptors;

(xiii) interleukins (ILs) and interleukin receptors, including but not limited to IL-1 to IL-15 and IL-1 to IL-15 receptors, such as the IL-8 receptor, among others;

(xiv) viral antigens, including but not limited to, an AIDS envelope viral antigen;

(xv) lipoproteins, calcitonin, glucagon, atrial natriuretic factor, natrecor (nesiritide; rh type B natriuretic peptide), lung surfactant, tumor necrosis factor-α and -β, enkephalinase, RANTES (regulated on activation normally T-cell expressed and secreted), mouse gonadotropin-associated peptide, DNAse, inhibin, and activin;

(xvi) integrin, protein A or D, rheumatoid factors, immunotoxins, bone morphogenetic protein (BMP), superoxide dismutase, surface membrane proteins, decay accelerating factor (DAF), AIDS envelope, transport proteins, homing receptors, addressins, regulatory proteins, immunoadhesins, antibodies;

(xvii) myostatins, TALL proteins, including TALL-1, amyloid proteins, including but not limited to, amyloid-beta proteins, thymic stromal lymphopoietins (“TSLP”), RANK ligand (“OPGL”), c-kit, TNF receptors, including TNF Receptor Type 1, TRAIL-R2, angiopoietins, and

(xvii) biologically active fragments or variants of any of the foregoing.

As to all of the foregoing, particularly contemplated are those proteins that are effective therapeutic agents, particularly those that exert a therapeutic effect by binding a target, particularly a target among those listed above, including targets derived therefrom, targets related thereto, and modifications thereof.

Each of these proteins is immobilized by non-covalent binding to a chromatography medium, such as an ion exchange medium. Ion exchange chromatography utilizes the interactions among the charged residues on a protein surface and the ligand or functional group that is immobilized on the beads. Typically, the ligand or functional group is covalently bound to the bead. Proteins that are bound to ion exchange beads effectively have their surface charge blocked. IgGs generally have a pI of around 7.5 to 8.5. At pH 5, e.g., IgG molecules bind to cation exchange beads. Furthermore, at pH above neutral, IgG molecules are not able to bind to cation exchange beads. The present disclosure provides for the use of cation exchange beads in a solid-state, stable formulation of IgG molecules useful for long-term storage in a pre-filled delivery vehicle such as syringes suitable for immediate use. A solid-phase formulation of a protein such as an antibody is prepared by binding the antibody drug product to an ion exchange medium such as cation exchange beads at, e.g., pH 5. The most commonly used formulation buffer for immunoglobulin G is 10 mM acetate, pH 5, and 5% sorbitol (A5S) and this buffer exemplifies the wide variety of buffers that can be used for the preparation of the solid-state formulations. Immediately prior to administration, and typically occurring as one of the initial events in such administration, the protein, e.g., antibody drug product, is eluted with buffer at pH 7 or higher. Phosphate-buffered saline, which is commonly used for administering drugs, is an exemplary elution buffer. At pH 7 or higher, the protein, e.g., antibody, will be uncharged and will not be able to bind to the beads. The advantages of this formulation include (1) a solid-phase formulation will restrict diffusion and improve storage stability even at room temperature, (2) neutralizing surface-charged residues by binding to ion exchange (e.g., cation exchange) media such as beads limits aggregation induced through salt bridges and ionic interactions, (3) the solid-state formulation is compatible with any injection route, such as intravenous, subcutaneous, and intraperitoneal administration, and (4) the formulation is useful for proteins susceptible to precipitation and instability at pH 5. Therapeutics according to the disclosure are proteins, such as antibodies, peptide hormones, growth factors, peptide agonists, peptide antagonists, and the like. Exemplary protein therapeutics include erythropoietin in any of its various forms including, but not limited to, Darbepoetin alfa (i.e., Aranesp®), as well as Etanercept (e.g., Enbrel®), Filgrastim or recombinant methionyl human granulocyte colony-stimulating factor (e.g., Neupogen®), and derivatives thereof, such as PEGylated forms of the protein therapeutics (e.g., Pegfilgrastim, e.g., Neulasta®). Other exemplary protein therapeutics include Herceptin® (trastuzumab), Trastuzumab-DM1 (a trastuzumab-DM1 conjugate), Avastin® (bevacizumab), Rituxan® (rituximab), Xolair® (omalizumab), Activase® (altiplase), TNKase® (Activase variant), Lucentis® (ranibizumab), Nutropin® (somatropin), Pulmozyme® (dornase alfa, rhDNase), Raptiva (efalizumab), Tarceva (erlotinib), ALTU-238, anti-CD20 antibody, anti-CD40 antibody, anti-IFN alpha, anti-beta7 integrin antibody, anti-OX40 ligand antibody, human APO2L/TRAIL, Apomab, BR3-Fc fusion protein, METMAb (anti-MET antibody), Pertuzumab, Remicade (infliximab), MabThera, Synagis (palivizumab), Humira, ReoPro (abciximab), efalizumab, alefacept, abatacept, infliximab, adalimumab, anti-TNFα antibodies, cytokines, anti-cytokine antibodies, In certain embodiments, protein therapeutics are provided that have a pI equal to or less than the pH of the elution fluid adsorbed to cation exchange media; also provided are protein therapeutics that have a pI equal to or greater than the pH of the elution fluid adsorbed to anion exchange media.

The pI, or isoelectric point, of a protein is readily determined empirically and those of skill in the art are aware of a variety of algorithms useful in estimating the pI of a protein from its amino acid sequence. An ion exchange resin typically is a solid, porous network (mineral or organic or composite) carrying ionizable groups of positive or negative charge and of a single group. Positively charged ionic groups (anion exchangers) include, for example, quaternary, tertiary and secondary amines and pyridine derivatives. Negatively charged ionic groups (cation exchangers) include, for example, sulfonates, carboxylates and phosphates. Selection of an ion exchange resin depends on the properties of the protein(s) to be bound. For amphoteric compounds such as proteins, the pI of the compound and its stability at various pH values determine the immobilization strategy. At a pH above its pI, the protein of interest will be negatively charged; at a pH below its pI the protein will be positively charged. Accordingly, if the protein is stable at a pH above its pI, an anion exchange resin is used. Conversely, if the protein is stable at a pH below its pI, a cation exchange resin is used. The operating pH also determines the type of exchanger to use. A strong ion exchange resin maintains capacity over a wide pH range, while a weak one loses capacity when the pH no longer matches the pKa of its functional group.

Anion exchangers can be classified as either weak or strong. The charge group on a weak anion exchanger is a weak base, which becomes deprotonated and, therefore, loses its charge at high pH. Diethyaminoethyl (DEAE)-cellulose is an example of a weak anion exchanger, where the amino group can be positively charged at a pH below about 9 and there is a gradual loss of charge at higher pH values. A strong anion exchanger, on the other hand, contains a strong base such as a quaternary amine, which remains positively charged throughout the pH range normally used for ion exchange chromatography (pH 2-12). Cation exchangers also can be classified as either weak or strong. A strong cation exchanger contains a strong acid (such as a sulfopropyl group) that remains charged from pH 1-14; a weak cation exchanger contains a weak acid (such as a carboxymethyl group), which gradually loses its charge as the pH decreases below about 4.5.

In some embodiments, strong ion exchangers, such as quaternary amines or sulfonic acids, are used. Weak ion exchangers, such as tertiary amines and carboxylic acids, also can be used, for example, when immobilizing a protein that has a pI between 5 and 8.

Chromatography media according to the disclosure include ion exchange media such as sepharose-, sepharose CL-, sepharose Fast Flow and sepharose High Performance-based ion exchange media, which consist of macroporous, beaded, cross-linked agarose to which charged groups are attached. The type of charged group determines the type and strength of the exchanger, while the total number and availability of the charged groups determine the capacity. Derivatizing any of these sorbents or base media to yield carboxymethyl groups creates a weak cation exchange medium, while derivatization to yield sulfopropyl or methyl sulfonate creates strong cation exchange media. Derivatization to create diethylaminoethyl (DEAE) groups creates a weak anion exchange material while derivatizing to yield quaternary aminoethyl (QAE) or quaternary ammonium (Q) creates strong anion exchange media. Many alternative ion exchange media are known in the art, any of which would be suitable for use in the compositions, delivery vehicles and methods according to the disclosure. By way of example, other known strong anion exchange media include UNO Q-1, Poros 50 HQ, Toyopearl QAE 550c; Separon HemaBio 1000Q, Q-Cellthru Bigbeads Plus and Toyopearl SuperQ 650s. In 1997, over 70 different ion exchange media were commercially available (Levison et al., J. Chromatogr. A 760:151-158 (1997), incorporated herein by reference), and choices have only expanded since that time.

Beyond ion exchange media, stable formulations of protein, e.g., protein therapeutics, may be achieved using hydrophobic interaction media. In this aspect, the loading and eluting fluids differ in ionic strength, with the eluting fluid having a lower ionic strength than the loading fluid. An exemplary eluting fluid for use with pre-filled vehicles comprising protein therapeutics immobilized to a hydrophobic interaction medium is phosphate-buffered saline. Any conventional loading buffer known to be useful in hydrophobic interaction chromatography (HIC) is contemplated as being useful in this aspect of the disclosed subject matter. Any eluting buffer known to be useful in HIC is also expected to be useful in this aspect of the disclosure; additionally, loading buffers modified to lower their ionic strength are also comprehended as eluting fluids useful in this aspect of the disclosure.

The chromatography medium may also be an affinity chromatography medium. In general, an affinity chromatography medium is a base substance to which is affixed, directly or indirectly, a compound (i.e., a binding partner) capable of specifically interacting with the protein to be bound to the chromatography medium, such as a protein therapeutic. In certain embodiments, the binding partner is a ligand for the protein to be bound. In practice, the protein to be bound to the chromatography medium will be partially or completely purified and, in such circumstances, the binding specificity of a binding partner need not be exclusive to the protein. Attachment of the binding partner to the chromatography base material may be covalent or non-covalent, provided that the binding partner will not substantially detach from the base material during contemplated use. Further, the binding partner may be directly affixed to the chromatography base material or it may be affixed through any linker, adaptor, or joining molecule known in the art, including but not limited to protein (e.g., peptide) molecules.

The binding capacity of a given ion exchange medium may be adjusted to any capacity within a broad range in view of the straightforward chemistry involved in derivatizing the base media used in manufacturing ion exchange media. The capacity of an ion exchange medium will be chosen depending on a number of variables known in the art and amenable to determination by those of skill in the art. For example, the binding capacity will be determined based on considerations that include the specific activity of a given protein therapeutic, the amount or range of activity in a therapeutic dose and the desired volume or range of desired volumes of a therapeutic dose.

The quantity, and hence volume, of chromatography medium 132 (see FIG. 1) non-covalently bound to a protein will define a bed volume of a pre-filled syringe according to the disclosure. The bed volume is associated with a void volume (i.e., volume of air or other fluid within the bed volume of chromatography medium) and that void volume is contemplated as being compatible with a volume of loading buffer that, upon delivery to an organism, e.g., a human patient, is insufficiently deleterious to outweigh the benefits of therapeutic delivery. In cases where loading buffers have relatively extreme pH and/or ionic strength characteristics, a wash solution may be applied to the pre-filled syringe following immobilization of the protein therapeutic. In general, such wash solutions are not expected to be necessary but those of skill in the art will recognize circumstances appropriate for post-immobilization application of a wash solution prior to elution of the protein therapeutic occurring as part of the delivery of that therapeutic.

Loading buffers contemplated to be compatible with maintaining a net charge on a given therapeutic that is opposite to the net charge on the ion exchange medium. The ionic strength of loading buffers can vary widely, provided that the ionic strength does not significantly interfere with the binding of the protein therapeutic to the ion exchange medium. In general, the ionic strength μ=1/2Σcᵢzᵢ² (where c is the charge of an ionic species i and z is the charge of that ion) of a loading buffer is expected to be less than or equal to the ionic strength of an elution buffer with which it is paired in preparing and using an immobilized form of a given therapeutic. Protein buffers suitable for use as loading buffers are generally prepared at a concentration of 1-200 mM buffer. Exemplary loading buffers are protein buffers, which include phosphate-buffered saline, phosphate buffers, CAPS (cyclohexylamino-1-propanesulfonic acid), CAPSO (cyclohexylamino-2-hydroxy-1-propane sulfonic acid), Cacodylate, Citrate salts, Glycine HCl, HEPES (N-[2-hydroxyethyl]piperazine-N′-[2-ethanesulfonic acid]), Imidazole, MES (morpholinoethanesulfonic acid), MOPS (3-[N-morpholino]-propanesulfonic acid), NEM (N-ethylmorpholine), PIPES (piperazine-1,4-bis-(2-ethanesulfonic acid)), Triethanolamine, Tris (HCl, acetate, sulfate), Bicine (bis-(2-hydroxyethyl)-glycine), TAPS (Tris-(hydroxymethyl)-methyl]-3-aminopropanesulfonic acid), TES (Tris-(hydroxymethyl)-methyl]-2-aminoethanesulfonic acid), Tricine (N-tris[hydroxymethyl]methylglycine), ACES (acetamido-2-aminoethanesulfonic acid), ADA (Acetamido-iminodiacetic acid), BES (bis-(2-hydroxyethyl)-2-aminoethane sulfonic acid), and any other buffer suitable for use with proteins or peptides that is known in the art. The range of pH within which protein buffers exhibit useful buffering capacity are known in the art (typically, within ±1 pH unit of the pKa of the compound used as a buffer) and will guide the selection of a buffer. Exemplary pH ranges for buffers are acetate (pH 4.2-5.2), MES (pH 5.5-6.9), HEPES (pH 6.8-8.2), and NEM (pH 7.2-8.5).

Elution buffers are biocompatible buffers having a combined pH and ionic strength sufficient to desorb, or elute, the therapeutic, preferably in a predictable manner, i.e., a manner wherein a given amount of therapeutic is reliably eluted upon passage of a given volume of eluting buffer. The chromatography medium provides an advantageous resistance to pressure-driven fluid flow, facilitating effective elution of non-covalently bound protein. Elution buffers have a pH, or ionic strength, sufficient to desorb a therapeutically effective amount of a protein therapeutic in an administrable volume of the buffer, as would be known in the art. Further, elution buffers for use with protein therapeutics immobilized on cation or anion exchange media preferably will have a pH that will modulate the charge on the protein and/or the media such that the protein can no longer bind to the media. Exemplary elution buffers include phosphate-buffered saline, phosphate buffers, CAPS, Citrate salts, Glycine HCl, HEPES, MES, MOPS, PIPES, Tris (HCl, acetate, sulfate), Bicine, Tricine, and any other buffer suitable for use with proteins or peptides that is known in the art. Another approach to protein elution would be to modulate the ionic strength of the elution buffer. Increased ionic strength (salt concentration) competes out the charge-charge interaction between the protein and the ion exchange medium. Hence, an increase in salt concentration leads to elution of the protein from the medium. Elution buffers generally will have an ionic strength greater than the loading buffer used in a given instance for methods, systems, delivery vehicles and kits designed to achieve protein therapeutic desorption by altering ionic strength because, in general, chromatography media have reduced binding capacity for a protein in a buffer of higher ionic strength. The volume of high ionic strength buffer contemplated is expected to be a relatively minor addition to the recipient subject, such as a mammal (e.g., a human) and is therefore not expected to result in an appreciable change in the ionic strength of the blood, or any other bodily fluid or tissue, sufficient to lead to a deleterious effect on health, such as an untoward change in osmotic pressure.

One suitable means of sterilization for the chromatography medium is by autoclave. Affinity chromatography materials, proteins (e.g., therapeutic proteins), delivery vehicles in the form of syringes or infusion modules, and packets may be sterilized by irradiation or by exposure to a fluid (liquid or gas) sterilization agent. Where proteins such as therapeutic proteins are exposed to such a fluid, that fluid will be chemically inert towards the protein. Exposure of a protein to radiation is controlled, as would be known in the art, with respect to type and level, such that the radiation does not produce unacceptable levels of chemical degradation of the protein. Unacceptable levels are those levels producing a detectable toxic effect in an organism or those that reduce the activity of a protein to ineffective levels in view of quantity, volume and cost considerations.

In typical applications of fractionating or separating mixtures of compounds, ion-exchange chromatography exploits the differing partitioning behaviors (between mobile and stationary phases) of the compounds that result from interactions between charged groups in the stationary phase and charges on the compounds found in the mobile phase. The stationary phase of an ion-exchange column may be a positively charged cation exchanger or a negatively charged anion exchanger. The charged groups are neutralized by oppositely charged counter ions in the mobile phase, the counter ions being replaced during chromatography by more highly charged sample molecules. It is preferable to use cross-linked columns, such as the cross-linked agarose of S-Sepharose Fast Flow™ cation exchange media. Alternatively, a membrane-based column could be employed. The column is usually washed after application of the protein therapeutic with any biocompatible buffer of relatively neutral pH (e.g., pH 6.5-7.5). An exemplary wash buffer is 20 mM HEPES buffer, pH 7.5. The antibody may be eluted with the same buffer containing physiological concentrations of sodium chloride (i.e., 0.154 M).

A mobile phase within the pH range of +/−1 pH unit away from the isoelectric point (pI) of the sample is suitable. For anion exchange columns, a mobile phase 1 pH unit above the isoelectric point of the sample is appropriate; for cation exchange media, a mobile phase 1 pH unit below the pI of the sample is effective.

The dosages of such antibodies will vary with the condition being treated and the recipient of the treatment, but will be in the range of about 1 to about 100 mg antibody protein therapeutic for an adult patient, preferably 1-10 mg, usually administered daily for a period between 1 and 30 days. A two-part dosing regime may be preferable, wherein 1-5 mg are administered for 5-10 days followed by 6-15 mg for a further 5-10 days.

Having provided a general description of the various aspects of the disclosed subject matter, the following disclosure provides illustrative examples, wherein Example 1 describes in vitro experiments, Example 2 discloses the results of studies assessing the effects of shear stress and Example 3 describes experiments for targeted administration of a protein therapeutic.

### EXAMPLES

### Example 1

### Stability

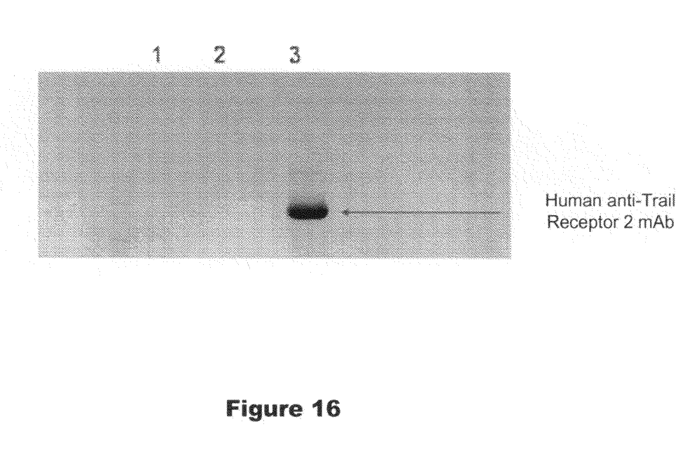

The solid-state formulation of protein therapeutics was demonstrated in vitro using the agonistic anti-TRAIL-R2 antibody described in connection with FIG. 16 (an antibody such as the antibodies described in provisional U.S. Ser. No. 60/713,433, filed Aug. 31, 2005, and provisional U.S. Ser. No. 60/713,478, filed Aug. 31, 2005, each of which is incorporated by reference herein), which is a fully human IgG anti-Trail Receptor 2 (TR-2) monoclonal antibody with a pI between 8.5 to 9. Materials used in conducting the experiments included trifluoroacetic acid (TFA), formic acid (FA) and guanidine hydrochloride (GdnHCl), which were obtained from Pierce (Rockford, Ill.). Dithiothreitol (DTT) and iodoacetamide (IAM) were obtained from Sigma-Aldrich (St. Louis, Mo.). HPLC grade water and acetonitrile (ACN) were obtained from VWR international (West Chester, Pa.). Pepsin and Trypsin were obtained from Roche'(Indianapolis, Ind.).

Reversed-phase chromatographic separation of IgG and IgG fragments was carried out on an Agilent 1100 HPLC system equipped with a Varian Diphenyl 2×150 mm column. A 20 mg protein sample was typically injected and elution was achieved with a linear A-B gradient for 40 minutes where eluent A was 0.1% aqueous trifluoroacetic acid (TFA) and eluent B was 0.1% TFA in 90% acetonitrile. The flow rate and temperature were maintained at 200 μl/minute and 75° C., respectively, throughout the run.

Reduction of IgG molecule was achieved by incubating 0.5 mL of IgG or IgG sample after limited proteolysis with LysC at a concentration of 2 mg/mL in denaturing buffer (7.5 M guanidine hydrochloride (GdnHCl), 120 mM sodium acetate, pH 5.0) containing 5 mM TCEP, at 37° C. for 30 minutes.

Ten mg of the agonistic anti-TRAIL-R2 antibody in A5S buffer (10 mM sodium acetate, pH 5.0, 5% sorbitol) were loaded on carboxymethyl (CM) Sepharose chromatography medium, which is a weak cation exchanger (WCX). Lane 1 of FIG. 16 shows a polyacrylamide gel electrophoretogram (PAGE analysis) of the flow-through fraction (i.e., fraction not bound by the medium). It can be seen that the flow-through fraction does not contain a significant amount of the band for the agonistic anti-TRAIL-R2 antibody, indicating that most of the loaded fraction was bound on the column. The medium was then washed with 10 ml of the pH 5 loading buffer. Lane 2 of the Figure represents the wash fraction. It can be seen from the Figure that the pH 5 wash fraction does not contain any agonistic anti-TRAIL-R2 antibody, demonstrating that, at pH 5, most of the protein is bound to the column. At pH 5, the protein has a positive charge while the WCX has a negative charge, leading to protein binding to the WCX medium. Lane 3 of FIG. 16 represents the fraction that was eluted from the medium with 1 M Tris HCl, pH 8. The combination of elevated pH (imparting a negative charge to the protein) and the high ionic strength of the Tris buffer led to the elution of the agonistic anti-TRAIL-R2 antibody, which was observed as a band on the gel. These data indicate that, at pH 5, the IgG is bound to the WCX medium and was readily eluted with buffers of higher pH and ionic strength. Thus, the method of providing a stable, storable form of protein therapeutics by immobilizing the protein to an ion exchange medium is functional because the ion exchange beads did bind and immobilize the protein therapeutic, that immobilization survived washing steps suitable for removing impurities, and the protein was quantitatively eluted using a physiologically compatible buffer.

The general applicability of the methods and delivery vehicles, and systems of the disclosure will be apparent to those of skill in the art upon review of the disclosure herein. Exemplifying this general applicability, Table 1 provides preferred conditions for preparing and using pre-filled vehicles containing any of a number of protein therapeutics. The ion exchange media listed in column 2 of Table 1 are defined in terms of the functional groups involved in ion exchange (e.g., carboxymethyl, sulfopropyl groups), which may be attached to any number of sorbents (e.g., sepharose, sephacryl, cellulose, trisacryl). Additional guidance on chromatography media, pH of loading buffer and pH of elution fluid suitable for proteins of a given pI is provided in Table 2.

TABLE 1

| Protein | Ion exchange | | pH of loading | pH of elution |
|---|---|---|---|---|
| Therapeutic | medium¹ | pI | buffer | fluid |
| IgG | CM WCX | 7.5-8.5 | 5.0 | 7.5-8.5 |
| Enbrel | SP SCX | 3.5-5.5 | 3.4 | 7.0 |
| Avimers | SP SCX | 4.0 | 3.5 | 7.0 |
| EPO | SP SCX | 4.5-5.3 | 4.0 | 7.0 |
| Aranesp | SP SCX | 4.3-4.5 | 4.0 | 7.0 |
| Neupogen/Neolasta | CM WCX | 6.02 | 5.0 | 7.0 |

¹CM is carboxymethyl, SP is sulfopropyl, WCX is weak cation exchange, and SCX is strong cation exchange.

TABLE 2

| Protein | Chromatography | Immobilization | Elution |
|---|---|---|---|
| pI range | medium | pH | pH |
| Above 7.0 | CM WCX | 5.0 | Above 7.0 |
| 5.5-7.0 | CM WCX | 5.0 | Above 5.0 |
| 3.5-5.5 | SP SCX | 3.4 | Above 5.0 |

### Example 2

### Shear Stress

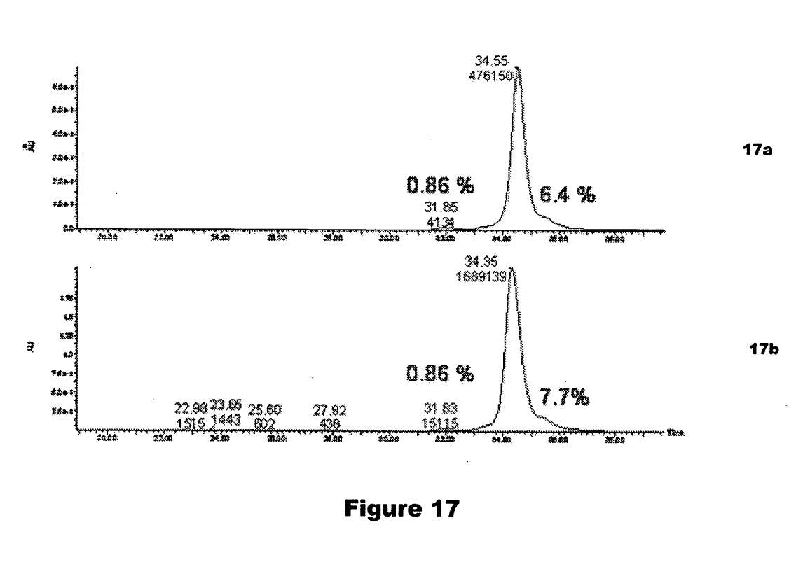

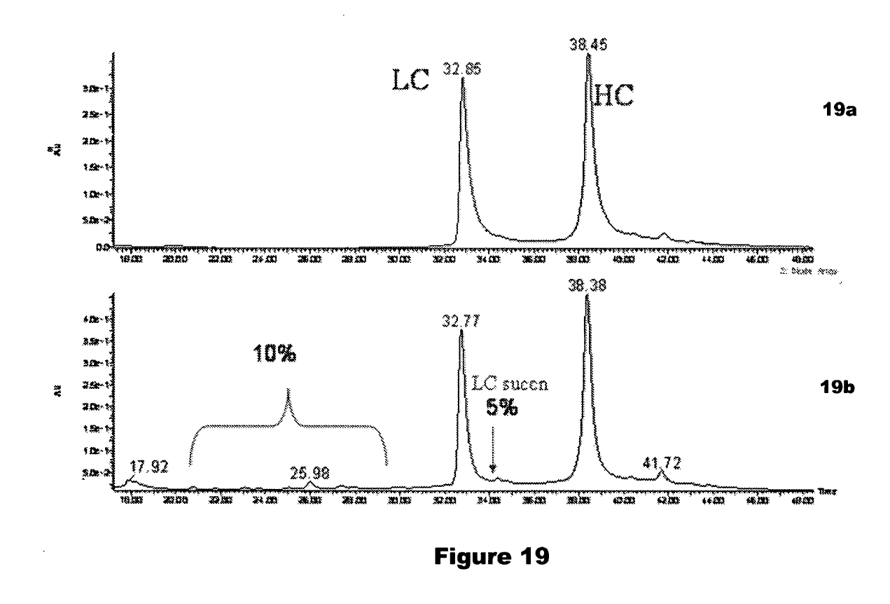

Short-term stability of the solid state formulation of the agonistic anti-TRAIL-R2 antibody buffered to pH 5 was compared to a liquid formulation in the same buffer. Two mg of the agonistic anti-TRAIL-R2 antibody were loaded onto one gram of carboxymethyl-sepharose and the resulting formulation medium was added to a 3 ml syringe. A corresponding 2 mg/ml liquid formulation was prepared in the same buffer as that used in the SSF, and added to 5 ml glass-stopper vials. Both these formulations were incubated at room temperature for 3 days on a shaker that was operated at 700 rpm. The formulations were compared using a variety of analytical techniques. FIG. 17 shows the reversed-phase chromatogram of the two formulations. Reversed-phase chromatography is a powerful protein separation technique that allows detection of protein degradation products such as fragments arising from peptide bond hydrolysis (i.e., clipping), as well as other chemical modifications of proteins. It can be seen from FIG. 17 that the two formulations yielded comparable reversed-phase chromatograms (the upper tracing FIG. 17a was the agonistic anti-TRAIL-R2 antibody bound to CM-sepharose; the lower tracing in FIG. 17b was a liquid formulation of the agonistic anti-TRAIL-R2 antibody). No major aggregation was observed in either of the formulations. The liquid formulation showed the presence of smaller fragments that were not very clearly distinguished in the reversed-phase chromatogram, but this issue is addressed by the results shown in FIG. 19.

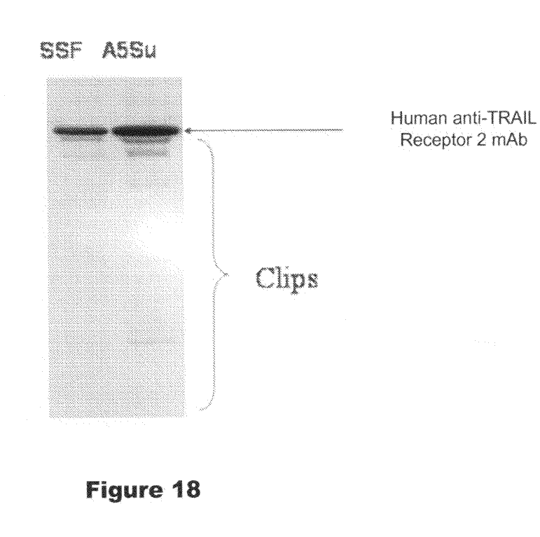

FIG. 18 shows the PAGE analysis of the two formulations. Both formulations show strong bands for the agonistic anti-TRAIL-R2 antibody, without any major covalent dimerization. Consistent with the chromatograms, the liquid formulations show more fragmentation. The data shown in FIG. 17a-b and 18 indicate that in short-term storage (e.g., three days at room temperature), the SSF formulation showed improved stability relative to the liquid formulation of this antibody.

To further analyze the fragmentation observed in the liquid formulation, reduced samples were analyzed by reversed-phase chromatography. Reduction reduces the complexity of molecules in the samples by separating the light and heavy chains that are linked together in intact, complete antibody molecules. Reduction also improves the resolution of the chromatographic assay. The reversed-phase chromatograms of the reduced samples from the two formulations are shown in FIG. 19, with the solid-state formulation shown in FIG. 19a and the liquid formulation shown in FIG. 19b. Two major peaks were observed in the chromatograms, which represent the light chain (LC) and heavy chain (HC) of the agonistic anti-TRAIL-R2 antibody. The liquid formulation also showed a post peak on the LC at levels of around 5% (FIG. 19b). Mass spectrophotometric analysis of the peak indicated a loss in mass of 17-18 kiloDaltons (kDa) in the post peak, which was caused by succinimide formation from asparagine or aspartic acid. Such chemical degradations sometimes lead to loss of biological activity. The solid-state formulations did not show a significant amount of the LC post peak, indicating that such a formulation could provide protection from chemical modifications. Solvent-exposed residues are usually more susceptible to chemical degradation. Without wishing to be bound by theory, interaction of the amino acid side chain with the chromatographic medium could restrict solvent accessibility, leading to a reduction in chemical degradation as compared to standard liquid formulations.

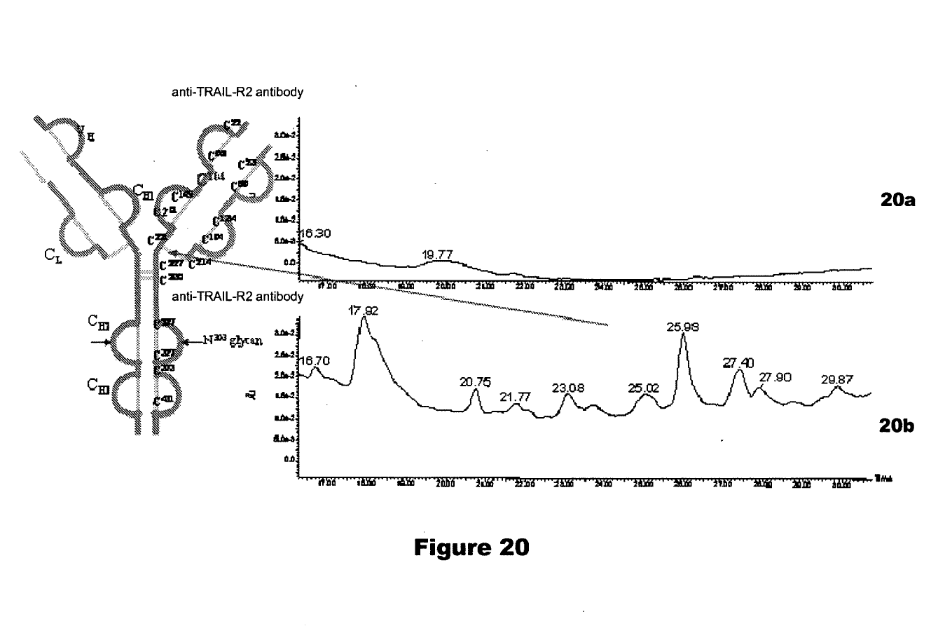

FIG. 19a-b also shows that the liquid formulation has additional peaks between retention times of 20 to 30 minutes. A detailed view of this region is shown in FIG. 20. It can be seen from FIG. 20 that the peaks observed in the liquid formulation are completely absent in the SSF. The peaks are caused by degradation (e.g., clipping) of the IgG molecule at the hinge region. Although not wishing to be bound by theory, it is known that the hinge region is susceptible to shear-induced hydrolysis or clipping. The SSF restricts motion and hence minimizes shear during shaking, thereby providing an immobilized protein with protection against shear-induced degradation. These data indicate that the SSF protects the bound protein from chemical degradation and physical degradation.

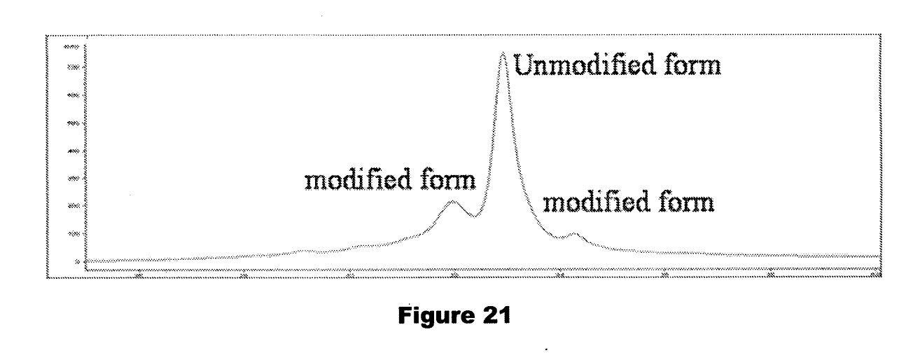

Analytical ion exchange is often used for the characterization of IgG molecules. Ion exchange separates charge variants in proteins. A weak cation exchange (WCX) separation of 146B7-CHO, an IgG1 molecule, is shown in FIG. 21. The elution for this experiment was carried out with a linear NaCl gradient. A major peak corresponding to the unmodified form is seen at 33 minutes. Peaks are observed on either side of the main peaks, and these additional peaks correspond to charge variants. These charge variants are caused by modifications such as deamidation and succinimide formation. Similarly, the pH and the ionic strength of the elution buffer for SSF may be adjusted by those of skill in the art using routine procedures in order to specifically deliver the unmodified form of protein.

The pH and ionic strength of the formulation buffer and the delivery/elution can be adjusted to provide for a SSF for any protein, e.g., protein therapeutic. Table 1 shows one example of how a combination of pH and cation exchange medium are used for SSF for proteins within a wide p1 range. Similarly, buffer pH and ionic strengths can be varied to make SSF compatible with anion exchange media as well as HIC media or affinity chromatography media.

The short-term shear stress study of a liquid formulation of the agonistic anti-TRAIL-R2 antibody and of the agonistic anti-TRAIL-R2 antibody non-covalently bound to CM-sepharose is presented in Table 3. The greater degradation seen in the liquid formulation relative to the SSF or formulation in which the agonistic anti-TRAIL-R2 antibody was non-covalently bound to CM-sepharose, is shown in the gel electrophoretogram of FIG. 18.

TABLE 3

| | Agonistic Anti- | | |
|---|---|---|---|
| | TRAIL-R2 | | |
| Name | Antibody STD | SSF | As5u |
| HMW Peak (%) | 0.37 | 0.39 | 0.26 |
| HMW (mAU · sec) | 437.112 | 63.645 | 114.775 |
| Main Peak (%) | 98.57 | 98.38 | 98.58 |
| Main Peak | 116763.428 | 16057.463 | 44117.706 |
| (mAU · sec) | | | |
| LMW Peak 1 (%) | 1.01 | 1.14 | 1.05 |
| LMW Peak 1 | 1198.325 | 188.219 | 476.197 |
| (mAU · sec) | | | |
| LMW Peak 2 (%) | 0.05 | 0.09 | 0.1 |
| LMW Peak 2 | 0.951 | 0.235 | 0.765 |
| (mAU · sec) | | | |

Table 3 catalogs the peaks and relative quantities under those peaks following size-exclusion chromatography. The results shown in FIG. 18 are consistent with the results provided in Table 3 in that FIG. 18 shows that fractionation of the samples following the three-day period of shaking revealed that the mobile protein therapeutic in solution (liquid formulation) was relatively labile in showing degradation (A5Su lane) whereas the immobilized protein therapeutic (protein non-covalently bound to CM-sepharose) did not show degradation (SSF lane). The results of this study, confirmed by the data provided below, establish that the immobilized protein therapeutics, particularly when packaged into the packed beds of pre-filled syringes, are more resistant to shear stress than free protein therapeutics during shipping and handling and do not suffer from adverse effects relative to those free protein therapeutics during shipping and handling, and indeed during any activity prior to administration, confirming advantages of the compositions, delivery vehicles, systems and methods of the disclosure.

The agonistic anti-TRAIL-R2 antibody subjected to short-term shear stress either in a liquid formulation or non-covalently bound to chromatography medium (CM-sepharose) was also reduced using conventional techniques to separate the heavy and light chains of the agonistic anti-TRAIL-R2 antibody, an antibody molecule. The reduced, separated antibody chains eliminated some complexity and provided a clearer picture of the fate of the agonistic anti-TRAIL-R2 antibody. The results provided in FIG. 19 indicated that the solid-state formulation of the agonistic anti-TRAIL-R2 antibody resulted in less degradation during short-term shear stress than the liquid formulation of the agonistic anti-TRAIL-R2 antibody. The graphs of FIG. 19a-b were analyzed more closely, with the more detailed view of the graphs being presented in FIG. 20a-b. The results of this study, as presented in FIGS. 19a-b and 20a-b, are consonant with the data already described in establishing the stability of proteins, such as therapeutics, immobilized in the packed beds of pre-filled delivery vehicles such as syringes.

### Example 3

### Targeted Drug Administration

The delivery vehicles of the disclosure are amenable to precise delivery of the desired form a protein therapeutic at the point-of-use. It is known that selection of buffer pH values near the pI of a given protein will facilitate the separation of the intact protein from fragments having even a slightly different pI than the holo-protein. This fact can be exploited in designing the pH of a loading buffer and/or an elution fluid to be near to the pI of the protein therapeutic. A loading buffer pH slightly more acidic than the pI of a protein therapeutic suitable for adsorption to a cation exchange medium may be chosen; analogously, a loading buffer pH slightly more alkaline than the pI of a protein therapeutic suitable for adsorption to an anion exchange medium may be chosen. In the alternative or in addition, an elution fluid of a pH slightly less acidic than the pI of a protein therapeutic could be selected for a protein therapeutic suitable for adsorption to a cation exchange medium while a pH slightly less alkaline than the pI of a protein therapeutic could be selected for an elution fluid used to desorb a protein therapeutic from an anion exchange medium.

Confirmation of the preceding observations was obtained by examining the ability of the presently disclosed system to separate the unmodified holo-protein form of 146B7-CHO from modified forms, of this protein therapeutic. These modified forms are typically charge variants arising from deamidation, succinimide formation, and the like. Such modifications are indications of protein instability, and often such modifications are associated with loss of activity. In the experiment, 146B7-CHO was loaded onto CM-sepharose, a weak cation exchange medium. The chromatography medium was washed using conventional procedures and non-covalently bound protein was eluted with a linear NaCl gradient. As shown in FIG. 21, the unmodified holo-protein form of 146B7-CHO can be distinguished from at least two modified forms of that protein by subjecting a sample of the protein to ion exchange chromatography, as would occur in loading and then eluting a protein therapeutic according to the disclosure. The ability to discriminate between an unmodified holo-protein and modified forms thereof indicates that the methods, systems, delivery vehicles and kits according to the disclosure are amenable to eluting conditions that specifically release the unmodified form of the protein. In addition, it is expected that the methods, systems, delivery vehicles and kits according to the disclosure will diminish or eliminate the modifications giving rise to modified forms of a protein associated with a loss or modification in activity. Thus, the subject matter disclosed herein will bring long-term, stable storage of proteins, including therapeutic proteins, to the medical and veterinary communities, and to individuals seeking self-treatment, by providing proteins in a form that facilitates reliable predictions of effective dosages applicable over considerable time periods.

Although the preceding text sets forth a detailed description of different embodiments of the invention, it should be understood that the legal scope of the invention is defined by the words of the claims set forth below. The detailed description is to be construed as exemplary only and does not describe every possible embodiment of the invention because describing every possible embodiment would be impractical, if not impossible. Numerous alternative embodiments could be implemented, using either current technology or technology developed hereafter, which would still fall within the scope of the claims defining the invention.

It should also be understood that, unless a claim element is defined by reciting the word “means” and a function without the recital of any structure, it is not intended that the scope of any claim element be interpreted based on the application of 35 U.S.C. §112, sixth paragraph. The entire disclosures of all publications cited herein are hereby incorporated by reference.

What is claimed is:

1. A system comprising:
(a) a delivery vehicle comprising
(i) at least one chamber in which is disposed a chromatography medium selected from the group consisting of a cation exchange medium, an anion exchange medium and a hydrophobic interaction medium, wherein the medium is non-covalently bound to at least one therapeutically effective dose of a protein therapeutic;
(ii) an inlet port; and
(iii) a medium restrictor for substantially preventing discharge of the medium from the delivery vehicle; and
(b) an elution fluid calibrated to release at least one therapeutically effective dose of the protein therapeutic.

2. The system according to claim 1 wherein the medium restrictor is selected from the group consisting of a filter and an outlet port.

3. The system according to claim 1 wherein the protein therapeutic is an antibody.

4. The system according to claim 1 wherein the medium is a cation exchange medium comprising a functional group selected from the group consisting of a carboxymethyl group, a sulfopropyl group and a methyl sulfonate group.

5. The system according to claim 1 wherein the delivery vehicle further comprises an in-line filter for preventing discharge of the medium from the chamber comprising the medium.

6. The system according to claim 1 wherein the delivery vehicle is a syringe.

7. The system according to claim 6 wherein the syringe comprises two chambers, wherein the medium is localized to one chamber.

8. The system according to claim 7 wherein the syringe further comprises a pressure-sensitive barrier separating the two chambers.

9. The system according to claim 7 wherein the medium is non-covalently bound to at least one therapeutically effective dose of a protein therapeutic.

10. A method of producing the system according to claim 1 comprising
(a) adding at least a predetermined quantity of the medium to the chamber comprising the medium, wherein the medium is non-covalently bound to a protein therapeutic; and
(b) determining the volume of an elution fluid to elute at least one therapeutically effective dose of the protein therapeutic.

11. A delivery vehicle comprising
(a) at least one chamber in which is disposed a chromatography medium selected from the group consisting of a cation exchange medium, an anion exchange medium, an affinity medium and a hydrophobic interaction medium, wherein the medium is non-covalently bound to at least one therapeutically effective dose of a protein therapeutic;
(b) an inlet port; and
(c) a medium restrictor for substantially preventing discharge of the medium from the delivery vehicle.

12. The delivery vehicle according to claim 11 wherein the medium restrictor is selected from the group consisting of a filter and an outlet port.

13. The delivery vehicle according to claim 11 wherein the protein therapeutic is an antibody.

14. The delivery vehicle according to claim 11 wherein the medium is a cation exchange medium comprising a functional group selected from the group consisting of a carboxymethyl group, a sulfopropyl group and a methyl sulfonate.

15. The delivery vehicle according to claim 11 wherein the delivery vehicle further comprises an in-line filter for preventing discharge of the medium from the chamber comprising the medium.

16. The delivery vehicle according to claim 11 wherein the delivery vehicle is a syringe.

17. The delivery vehicle according to claim 16 wherein the syringe comprises two chambers, wherein the medium is localized to one chamber.

18. The delivery vehicle according to claim 17 wherein the syringe further comprises a pressure-sensitive barrier separating the two chambers.

19. The delivery vehicle according to claim 17 wherein the medium is non-covalently bound to at least one therapeutically effective dose of a protein therapeutic.

20. A method of administering a protein therapeutic to a subject comprising:
(a) contacting a medium non-covalently bound to at least one therapeutically effective dose of a protein therapeutic with an elution fluid, wherein the medium is confined in one chamber of a syringe or infusion module comprising at least one chamber;
(b) eluting at least one therapeutically effective dose of the protein therapeutic; and
(c) discharging the eluted protein therapeutic from the syringe or infusion module, thereby administering a therapeutically effective dose of the protein therapeutic to the subject.

21. The method according to claim 20 wherein the protein therapeutic is an antibody.

22. The method according to claim 20 wherein the contacting step comprises rupturing a fluid-impermeable barrier covering the inlet port of the chamber comprising the medium.

23. The method according to claim 22 wherein the rupturing is accomplished by applying fluid pressure to the membrane by actuating a syringe plunger comprising a head member sealingly engaged with the internal surface of the syringe.

24. A kit for administering a protein comprising an infusion module or syringe, wherein the infusion module or syringe comprises a chromatography medium non-covalently bound to a protein, and a package insert for providing instruction on the use thereof.
